SEQUENCE LISTING (39 sequences)

SEQ ID NO: 1 - protein, 14 aa, Aspergillus niger
KSGSGIDNLLSDER

SEQ ID NO: 2 - DNA, 21 bp, Artificial Sequence
  Forward primer to amplify genomic sequence of the newly identified amine oxidase.
cacttcctcagcctaatttgc

SEQ ID NO: 3 - DNA, 21 bp, Artificial Sequence
  Reverse primer to amplify genomic sequence of the newly identified amine oxidase.
ctggtgtagatctaacgaata

SEQ ID NO: 4 - DNA, 1395 bp, Aspergillus niger
atgtctgtatccaacgatcctactacaaagctctacgatgcggtgatcgtgggagccggacttagcggccttcaagccgcgcattccatccaggcagcaggattcagcgtgtgtatcctggaagctacggaccgaatgggtgggaagacgttgaccgtaaaatccagcgagaaaggatacaacgatctaggagcggcttgggtgaatgatacgaaccagacggagattttcaaacttcatcagcggtatggactggatggggtggttcagtatacttgtggagatgatatcctcgagtcaggcgagggggtgatccgcaagataccgtatggattgccattgactgggcttccgaagaaattgttggatattctccgaattgagtcctcacggttagacttggacgatcctacgagttttccaggggccacagaagtggataatctgacgttcagggacttttgcgtcgagaagactggatcggaggatgttattcacattacagatgctatttcaacggcgctgcttggattgaatagtaacgaactcagtgctttgtatatgctctactacttcaaaagtgggagtgggatcgacaatctgctgtcagatgagagagacggagcacagtacctgcggacaagacaaggtacccaaaccatcgcccggaagatggcagatgagctcacccaatcggacatttttctgggcatgcccgtcacttcaatcaatcagactgacgctgatgctcactgcgtggtccagacacttgatggaagttcttttcgctgtcgacgcgttatcgtgtctatccctaccaccttataccggagtgtctccttccaccccccacttccacatgcaaaacaggtattaagtgaccatacgatcatgggatactacagtaaagtgatcttcatcttcaaagaaccatggtggcgcgacgctggacttaccggaatcgtcaactgtgcgggtggccccataacctttacacgggacacgagtgtacccaccgacgaccaatggtctatcacatgcttcatggtgggcagtcgcggacgagcgtggtctaagctgtcaaaggatgatcgatacagccaagtgtgggagcagtttcgccgatgttttgaagagttcgtggaaaacatccccgagccagtaaataccctggagatggaatggagtaaagagccttatttccttggagcaccttgtccggctatgatacccggcttactgactactgccgggagtgatctagctgcaccgcacggcaaagtgcattttatcggaacagaaacgtccacagtgtggcgtgggtacatggaaggggctattagagccgggcagcgaggaggagccgaggttgtgacggcactgcaggaagactag

SEQ ID NO: 5 - protein, 464 aa, Aspergillus niger
MSVSNDPTTKLYDAVIVGAGLSGLQAAHSIQAAGFSVCILEATDRMGGKTLTVKSSEKGYNDLGAAWVNDTNQTEIFKLHQRYGLDGVVQYTCGDDILESGEGVIRKIPYGLPLTGLPKKLLDILRIESSRLDLDDPTSFPGATEVDNLTFRDFCVEKTGSEDVIHITDAISTALLGLNSNELSALYMLYYFKSGSGIDNLLSDERDGAQYLRTRQGTQTIARKMADELTQSDIFLGMPVTSINQTDADAHCVVQTLDGSSFRCRRVIVSIPTTLYRSVSFHPPLPHAKQVLSDHTIMGYYSKVIFIFKEPWWRDAGLTGIVNCAGGPITFTRDTSVPTDDQWSITCFMVGSRGRAWSKLSKDDRYSQVWEQFRRCFEEFVENIPEPVNTLEMEWSKEPYFLGAPCPAMIPGLLTTAGSDLAAPHGKVHFIGTETSTVWRGYMEGAIRAGQRGGAEVVTALQED

SEQ ID NO: 6 - DNA, 1395 bp, Artificial Sequence
  AnFAO 15309 codon optimized nucleotide sequence for expression in E. coli
atgagcgtgtctaatgatccgacaaccaaactgtatgatgcagttattgttggtgccggtctgtctggtctgcaggccgcccattctattcaggcagccggctttagcgtgtgtattctggaagcaaccgatcgtatgggtggtaaaaccctgaccgttaaatctagcgaaaaaggctataatgatctgggtgcagcatgggttaatgataccaatcagaccgaaatttttaagctgcatcagcgctatggtctggatggcgttgttcagtatacctgtggtgacgatattctggaaagcggtgaaggtgtgattcgtaaaattccgtatggcctgccgctgaccggtctgccgaaaaaactgctggatattctgcgcattgaaagctctcgtctggatctggatgatccgacaagctttccgggtgccaccgaagttgataatctgacctttcgtgatttttgtgttgaaaaaaccggctctgaagatgtgattcatattaccgatgcaatttctacagcactgctgggcctgaatagtaatgaactgagcgcactgtatatgctgtattattttaaaagcggcagcggcattgataatctgctgtctgatgaacgcgatggtgcacagtatctgcgtacccgtcagggcacccagaccattgcacgcaaaatggcagatgaactgacacagagcgatatttttctgggtatgccggttacatctattaatcagaccgatgcagatgcacattgtgttgtgcagacactggatggctcttcttttcgctgtcgccgcgtgattgtgtctattccgaccaccctgtatcgcagcgtgagctttcatccgccgctgccgcatgcaaaacaggtgctgagcgatcatacaattatgggttattatagcaaggttattttcatcttcaaggaaccgtggtggcgtgatgcaggtctgaccggtattgtgaattgcgccggtggtccgattacctttacccgtgatacatctgtgccgacagatgatcagtggagcattacctgttttatggttggtagccgcggccgtgcatggtctaaactgagcaaagatgatcgctatagccaggtttgggaacagtttcgccgttgctttgaagaatttgtggaaaatattccggaaccggttaataccctggaaatggaatggagcaaagaaccgtattttctgggtgcaccgtgtccggcaatgattccgggtctgctgaccacagccggctctgatctggccgcaccgcatggcaaagtgcattttattggcaccgaaacctctacagtgtggcgtggctatatggaaggtgccattcgtgcaggtcagcgtggcggcgcagaagtggttaccgcactgcaggaagattaa

SEQ ID NO: 7 - protein, 19 aa, Artificial Sequence
  invertase signal sequence
MLLQAFLFLLAGFAAKISA

SEQ ID NO: 8 - protein, 18 aa, Artificial Sequence
  S. cerevisiae AGA2 signal sequence
MQLLRCFSIFSVIASVLA

SEQ ID NO: 9 - protein, 21 aa, Artificial Sequence
  S. cerevisiae alpha mating factor
MRFPSIFTAVLFAASSALAAP

SEQ ID NO: 10 - protein, 268 aa, Artificial Sequence
  SED 1 tethering moiety
KDNSSTIEGRYPYDVPDYALQASGGGGSGGGGSGGGGSASALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLF

SEQ ID NO: 11 - protein, 129 aa, Artificial Sequence
  SPI1 tethering moiety
LVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 12 - protein, 112 aa, Artificial Sequence
  CCW12 tethering moiety
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 13 - protein, 112 aa, Artificial Sequence; identical to SEQ ID NO: 12
  CCWP2 tethering moiety

SEQ ID NO: 14 - protein, 252 aa, Artificial Sequence
  TIR1 tethering moiety
KDNSSTIEGRYPYDVPDYALQASGGGGSGGGGSGGGGSASSLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 15 - protein, 465 aa, Artificial Sequence
  PST1 tethering moiety
KDNSSTIEGRYPYDVPDYALQASGGGGSGGGGSGGGGSASATSSSSSIPSSCTISSHATATAQSDLDKYSRCDTLVGNLTIGGGLKTGALANVKEINGSLTIFNATNLTSFAADSLESITDSLNLQSLTILTSASFGSLQSVDSIKLITLPAISSFTSNIKSANNIYISDTSLQSVDGFSALKKVNVFNVNNNKKLTSIKSPVETVSDSLQFSFNGNQTKITFDDLVWANNISLTDVHSVSFANLQKINSSLGFINNSISSLNFTKLNTIGQTFSIVSNDYLKNLSFSNLSTIGGALVVANNTGLQKIGGLDNLTTIGGTLEVVGNFTSLNLDSLKSVKGGADVESKSSNFSCNALKALQKKGGIKGESFVCKNGASSTSVKLSSTSKSQSSQTTAKVSKSSSKAEEKKFTSGDIKAAASASSVSSSGASSSSSKSSKGNAAIMAPIGQTTPLVGLLTAIIMSIM

SEQ ID NO: 16 - protein, 109 aa, Artificial Sequence
  AGA1/AGA2 tethering moiety
KDNSSTIEGRYPYDVPDYALQASGGGGSGGGGSGGGGSASQELTTICEQIPSPTLESTPYSLSTTTILANGKAMQGVFEYYKSVTFVSNCGSHPSTTSKGSPINTQYVF

SEQ ID NO: 17 - protein, 127 aa, Artificial Sequence
  AGA1/AGA2 tethering moiety
MQLLRCFSIFSVIASVLAQELTTICEQIPSPTLESTPYSLSTTTILANGKAMQGVFEYYKSVTFVSNCDSHPSTTSKDSPINTQYVFKDNSSTIEGRYPYDVPDYALQASGGGGSGGGGSGGGGSAS

SEQ ID NO: 18 - protein, 546 aa, Artificial Sequence
  FLO1 tethering moiety
KDNSSTIEGRYPYDVPDYALQASGGGGSGGGGSGGGGSASIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSESSKSSVIPTSSSTSGSSESETSSAGSVSSSSFISSESSKSPTYSSSSLPLVTSATTSQETASSLPPATTTKTSEQTTLVTVTSCESHVCTESISPAIVSTATVTVSGVTTEYTTWCPISTTETTKQTKGTTEQTTETTKQTTVVTISSCESDVCSKTASPAIVSTSTATINGVTTEYTTWCPISTTESRQQTTLVTVTSCESGVCSETASPAIVSTATATVNDVVTVYPTWRPQTANEESVSSKMNSATGETTTNTLAAETTTNTVAAETITNTGAAETKTVVTSSLSRSNHAETQTASATDVIGHSSSVVSVSETGNTKSLTSSGLSTMSQQPRSTPASSMVGYSTASLEISTYAGSANSLLAGSGLSVFIASLLLAII

SEQ ID NO: 19 - protein, 15 aa, Artificial Sequence
  (G4S)3 linker
GGGGSGGGGSGGGGS

SEQ ID NO: 20 - protein, 8 aa, Artificial Sequence
  (G)8 linker
GGGGGGGG

SEQ ID NO: 21 - protein, 40 aa, Artificial Sequence
  (G4S)8 linker
GGGGSGGGGSGGGGSGGGGSGGGGSGGGGSGGGGSGGGGS

SEQ ID NO: 22 - protein, 12 aa, Artificial Sequence
  linker
GSAGSAAGSGEF

SEQ ID NO: 23 - protein, 12 aa, Artificial Sequence
  (EAAK)3 linker
EAAKEAAKEAAK

SEQ ID NO: 24 - protein, 20 aa, Artificial Sequence
  (AP)10 linker
APAPAPAPAPAPAPAPAPAP

SEQ ID NO: 25 - protein, 46 aa, Artificial Sequence
  A(EAAAK)4ALEA(EAAAK)4A  linker
AEAAAKEAAAKEAAAKEAAAKALEAEAAAKEAAAKEAAAKEAAAKA

SEQ ID NO: 26 - protein, 22 aa, Artificial Sequence
  HA tag linker
KDNSSTIEGRYPYDVPDYALQA

SEQ ID NO: 27 - protein, 464 aa, Aspergillus niger
MSVSNDPTTKLYDAVIVGAGLSGLQAAHSIQAAGFSVCILEATDRVGGKTLTVKSSEKGYNDLGAAWVNDTNQTEIFKLHQRYGLDGVVQYTCGDDILESGEGVIRKIPYGLPLTGLPKKLLDILRIESSRLDLDDPTSFPGATEVDNLTFRDFCVEKTGSEDVIHITDAISTALLGLNSNELSALYMLYYFKSGSGIDNLLSDERDGAQYLRTRQGTQTIARKMADELSQSDIFLGMPVTSINQTDADAHCVVKTLDGSSFRCRRVIVSIPTTLYRSVSFHPPLPHAKQVLSDHTIMGYYSKVIFIFKEPWWRDAGLTGIVDCAGGPITFTRDTSVPTDDQWSITCFMVGSRGRAWSKLSKDDRYSQVWEQFRRFFEEFVENIPEPANTLEMEWSKEPYFLGAPCPAMIPGLLTTAGSDLAAPHGKVHFIGTETSTVWRGYMEGAIRAGQRGGAEVVTALQED

SEQ ID NO: 28 - protein, 464 aa, Aspergillus niger
MSVSNDPTTKLYDAVIVGAGLSGLQAAHSIQAAGFSVCILEATDRVGGKTLTVKSSEKGYNDLGAAWVNDTNQTEIFKLHQQYGLDGVVQYTCGDDILESGEGVIRKIPYGLPLTGLPKKLLDILRIESSRLDLDDPTSFPGATEVDNLTFRDFCVEKTGSEDVIHITDAISTALLGLNSNELSALYMLYYFKSGSGIDNLLSDERDGAQYLRTRQGTQTIARKMADELTQSDIFLGMPVTSINQTDADAHCVVQTLDGSSFRCRRLIVSIPTTLYRSVSFHPPLPHAKQVLSDHTIMGYYSKVIFIFKEPWWRDAGLTGIVNCAGGPITFTRDTSVPTDDQWSITCFMVGSRGRAWSKLSKDDRYSQVWEQFRRCFEEFVENIPEPVNTLEMEWSKEPYFLGAPCPAMIPGLLTTAGSDLAAPHGKVHFIGTETSTVWRGYMEGAIRAGQRGGAEVVTALQED

SEQ ID NO: 29 - protein, 464 aa, Aspergillus niger
MSVSNDPTTKLYDAVIVGAGLSGLQAAHSIQAAGFSVCILEATDRVGGKTLTVKSSEKGYNDLGAAWVNDTNQTEIFKLHQQYGLDGVVQYTCGDDILESGEGVIRKIPYGLPLTGLPKKLLDILRIESSRLDLDDPTSFPGATEVDNLTFRDFCVEKTGSEDVIHITDAISTALLGLNSNELSALYMLYYFKSGSGIDNLLSDERDGAQYLRTRQGTQTIARKMADELTQSDIFLGMPVTSINQTDADAHCVVQTLDGSSFRCRRVIVSIPTTLYRSVSFHPPLPHAKQVLSDHTIMGYYSKVIFIFKEPWWRDAGLTGIVDCAGGPITFTRDTSVPTDDQWSITCFMVGSRGRAWSKLSKDDRYSQVWEQFRRCLEGFVENIPEPANTLEMEWSKEPYFLGAPCPAMIPGLLTTTGSDLAAPHGKVHFIGTETSTVWRGYMEGAIRAGQRGGAEVVTALQED

SEQ ID NO: 30 - protein, 464 aa, Aspergillus niger
MSVSNDPTTKLYDAVIVGAGLSGLQAAHSIQAAGFSVCILEATDRVGGKTLTVKSSEKGYNDLGAAWVNDTNQTEIFKLHQQYGLDGVVQYTCGDDILESGEGVIRKIPYGLPLTGLPKKLLDILRIESSRLDLDDPTSFPGATEVDNLTFRDFCVEKTGSEDVIHITDAISTALLGLNSNELSALYMLYYFKSGSGIDNLLSDERDGAQYLRTRQGTQTIARKMADELSQSDIFLGMPVTSINQTDADAHCVVQTLDGSSFRCRRVIVSIPTTLYRSVSLHPPLPHAKQVLSDHTIMGYYSKVIFIFKEPWWRDAGLTGIVDCAGGPITFTRDTSVPTDDQWSITCFMVGSRGRAWSKLSKDDRYSQVWEQFRRCFEEFVENIPEPVNTLEMEWSKEPYFLGAPCPAMIPGLLTTAGSDLAAPHGKVHFIGTETSTVWRGYMEGAIRAGQRGGAEVVTALQED

SEQ ID NO: 31 - protein, 464 aa, Aspergillus niger
MSVSNDPTTKLYDAVIVGAGLSGLQAAHSIQAAGFSVCILEATDRVGGKTLTVKSSEKGYNDLGAAWVNDTNQTEIFKLHQRYGLDGVVQYPCGDDILESGEGVIRKIPYGLPLTGLPKKLLDILRIESSRLDLDDPMSFPGATEVDNLTFRDFCVEKTGSEDVIHITDAISTALLGLNSNELSALYMLYYFKSGSGIDNLLSDERDGAQYLRTRQGTQTIARKMADELSQSDIFLGMPVTSINQTDADAHCVVQTLDGSSFRCRRVIVSIPTTLYRSVSFHPPLPHAKQVLSDHTIMGYYSKVIFIFKEPWWRDAGLTGIVDCAGGPITFTRDTSVPTDDQWSITCFMVGSRGRAWSKLSKDDRYSQVWEQFRRCFEDFVENIPEPANTLEMEWSKEPYFLGAPCPAMIPGLLTTAGSDVAAPHGKVHFIGTETSTVWRGYMEEAIRAGQRGGAEVVTALQED

SEQ ID NO: 32 - protein, 464 aa, Aspergillus niger; identical to SEQ ID NO: 31

SEQ ID NO: 33 - DNA, 1794 bp, Aspergillus niger
atggctccagcacctgtcctggccacctccgcctactacgcccccggcgtggtgtcatccgcctccaaatatctccatgtctcgggccaaccaggcaccattgaaggcactgcccccgccgactacaattcgcagatccatcttgccctcgtgaatttgcatcgtgtgctagccgccaccggcgctacaccccgcgatgtagtcaaactcaccctctacatcgtcgactatgaccccaacaaccgtctacacactcgacccctgcagacatggctagctggccacaaaccagccattactcttgttccagtacctcaactagcagttccggactggaaattcgaaatcgaagccaccgtcgcggtgcctgactccattccagcttcgctctctcttccttcccccacagaaacgactgatgttctcgtcattggcgccggcctgtctggcctcatggccgccgagaccaccctccagtccggtcactcgtgcattgtgctcgaaggccgcgaccgcgtgggcggcaagacctggacttgtccactgccgagtggcacaggagtggttgatctcggcgccgcctggatcaacgataccaaccagagcatgatgtatgagttggcgcgacgggcaggggccgatttgatcgagcagaacaccacgggcaactgtcttttgcagcgggaggacggtgccattactgcttttccttacgggcagactccatgcataaccccccaaattgtaaaagaaatagaagccatccgcgacaccgccgagcaagactgccaatccttttctaccagtcgtccccagagtcccgctctcgactctctctctttcctcgcctatctgcattcccgcaacgccagccccatcgccgctgccaacgcctccgtctggactcgcgccatgcttggacaggaaccccaggacatctccgccctctactttctcaattactgcaagtccggcggcggattactccagatgcgctcggatcggaaacacggcgcgcagtatctacgtgtgcgacagggaacgcagatatttgcgaagactttggcggaatctttgcctacggatacaattcggttcggtcagagagtggtagggatcacgcaggtgcagaaaggggtgaattacgtgcagacggagagtggattggtggtcaaggctcggaaggtgatttgttctgtgccgacacctgtcctcaagacgatcaagttcgagccgcaactacctgctgccaagcagctcctggtcgattctttcagatatggctactacactaaggtgatgttgtcgttccgtacagcgtggtgggcggatcgtgggttctgtggactggcacagtcgtttgttggtcctgcatcgatttatcgcgatacgagtagcccagaggatgggaagtgggtgctaacggcgtttttggcgggtgatgctgggaggaactggtctgcgttggggagtcaaagggagcgtgaattggcgttgttggagcagttgggggctatctatggcgataaggatctgcctaagagggagtttgtcgaggctctcggtcatgagtggtctactgaggagctctctggctggggctgtccttgtccggcactgccgccgggcgtgttgacgctggctggagatgctctccgagaaccgtttcgggatgtgcatttcgtggggacagagacggcagaggagtggaagggttatatggagggtgcagtgcgcagtgggaagaggggggcggcggaggctgttaaggggctgacgaggagtcaattgtga

SEQ ID NO: 34 - DNA, 21 bp, Artificial Sequence
  AORIDF1 - Forwad primer
aagtcaacacttccccgcacg

SEQ ID NO: 35 - DNA, 21 bp, Artificial Sequence
  AORIDR1 - Reverse primer
tatagcacgagtgcctcggaa

SEQ ID NO: 36 - protein, 597 aa, Aspergillus niger
MAPAPVLATSAYYAPGVVSSASKYLHVSGQPGTIEGTAPADYNSQIHLALVNLHRVLAATGATPRDVVKLTLYIVDYDPNNRLHTRPLQTWLAGHKPAITLVPVPQLAVPDWKFEIEATVAVPDSIPASLSLPSPTETTDVLVIGAGLSGLMAAETTLQSGHSCIVLEGRDRVGGKTWTCPLPSGTGVVDLGAAWINDTNQSMMYELARRAGADLIEQNTTGNCLLQREDGAITAFPYGQTPCITPQIVKEIEAIRDTAEQDCQSFSTSRPQSPALDSLSFLAYLHSRNASPIAAANASVWTRAMLGQEPQDISALYFLNYCKSGGGLLQMRSDRKHGAQYLRVRQGTQIFAKTLAESLPTDTIRFGQRVVGITQVQKGVNYVQTESGLVVKARKVICSVPTPVLKTIKFEPQLPAAKQLLVDSFRYGYYTKVMLSFRTAWWADRGFCGLAQSFVGPASIYRDTSSPEDGKWVLTAFLAGDAGRNWSALGSQRERELALLEQLGAIYGDKDLPKREFVEALGHEWSTEELSGWGCPCPALPPGVLTLAGDALREPFRDVHFVGTETAEEWKGYMEGAVRSGKRGAAEAVKGLTRSQL

SEQ ID NO: 37 - DNA, 1395 bp, Artificial Sequence
  AnFAO_15309 codon optimized for Saccharomyces cerevisiae
atgagtgtctctaatgatccaactacgaaattgtacgacgcggtcattgtcggtgcgggacttagcgggcttcaagccgcgcattcaatccaagcggcgggttttagcgtctgcatattggaggcaactgatcgtatgggaggaaagactctaactgtaaagagttcagagaaggggtacaatgacctgggtgcggcatgggtaaacgatacgaatcagacggagattttcaagcttcaccaaaggtacggtttagatggtgttgtccaatatacctgtggggacgacatactggaatccggggagggggttattagaaaaataccgtatggattgccgttgacagggttgccgaagaagttgcttgatatattaagaatagagtcttctcgtttggatttagacgaccctacctcttttccgggcgctactgaggttgacaacttaacgttcagggatttctgtgttgagaaaacggggagtgaagatgtgatacatatcaccgacgcgatatccaccgccctgctaggcttgaactctaacgaactttcagcattatacatgctgtattactttaagtcaggatcaggtattgacaatctattgtccgatgaaagagacggagcccagtatctaaggaccagacaagggactcaaactatagctcgtaaaatggcagacgaattaacccaatccgatatattcctagggatgccagtcacatccattaatcagaccgacgctgatgcgcattgtgtggtacagacattagatggaagctcctttagatgtaggagagttattgttagtatacccacgacgctttacagaagtgtttccttccaccctcctttgccacatgccaagcaagttcttagcgatcacacgataatgggatactattccaaggtcatattcattttcaaggaaccttggtggcgtgacgcgggtcttacgggaatagtaaactgcgcagggggcccgattacctttactagagatacttccgtaccgacggatgatcaatggtcaataacatgctttatggtggggagtagaggtagagcgtggagcaagctttcaaaagacgatcgttattcccaggtgtgggaacaattcaggagatgttttgaagaattcgtcgagaacattccggagccggttaatacacttgagatggaatggagtaaggaaccctatttccttggtgccccctgcccggccatgattcccggccttctgacaaccgcgggctccgatctagcagcgccccacggcaaggtacattttataggcaccgaaacttccacagtgtggcgtggctatatggagggcgcgatcagagccggacagagagggggtgccgaggtggtaacggctctgcaagaagattaa

SEQ ID NO: 38 - protein, 6 aa, Artificial Sequence
  Hexa peptide motif sequence
GAGLSG

SEQ ID NO: 39 - protein, 6 aa, Artificial Sequence
  Hexa peptide motif sequence
  VARIANT (1)...(6): Xaa = Any Amino Acid
GXGXXG

(19) United States
(12) Patent Application Publication
(10) Pub. No.: US 2021/0337838 A1
(43) Pub. Date: Nov. 4, 2021

(54) RECOMBINANT EXPRESSION OF FUMONISIN AMINE OXIDASE

(71) Applicant: HER MAJESTY THE QUEEN IN RIGHT OF CANADA, AS REPRESENTED BY THE MINISTER OF AGICULTURE AND AGRI FOOD, Ottawa (CA)

(72) Inventors: Christopher Peter Garnham, London (CA); Mark William Sumarah, London (CA); Justin Beneteau Renaud, London (CA); Patrick Gordon Telmer, London (CA); Shane Gordon Butler, Cambridge (CA)

(21) Appl. No.: 17/273,660

(22) PCT Filed: Sep. 4, 2019
(86) PCT No.: PCT/CA2019/051230

§ 371 Date: Mar. 4, 2021

Related U.S. Application Data
(60) Provisional application No. 62/727,217, filed on Sep. 5, 2018.

(51) Int. Cl. A23L 5/20; A21D 8/04; A23K 10/38; A23K 20/189

(52) U.S. Cl. CPC A23L 5/25; A23K 20/189; A23K 10/38; A21D 8/047

(57) ABSTRACT

Fumonisins are a type of mycotoxin that contaminate different products, for example, feed and food products, including corn-based products, which can lead to serious health risks to humans and livestock. Current methods for detoxifying fumonisin-contaminated products are complex and expensive. The present disclosure provides a recombinant microbial host cell expressing an heterologous polypeptide having fumonisin amine oxidase activity, the recombinant microbial host cell comprising an heterologous nucleic acid molecule encoding the heterologous polypeptide having fumonisin amine oxidase activity, a variant thereof or a fragment thereof. The heterologous polypeptide having fumonisin amine oxidase activity can be used to detoxify a fumonisin mycotoxin present in feed and food products, for example from grains and products derived from grains.

### CROSS-REFERENCE TO RELATED APPLICATIONS AND DOCUMENTS

The present application claims priority from U.S. provisional application 62/727,217 filed Sep. 5, 2018 and herewith incorporated in its entirety. The present application includes a sequence listing entitled 55729550-41PCT_Sequence listing as filed which is also incorporated in its entirety.

### TECHNOLOGICAL FIELD

The present disclosure concerns recombinant fumonisin amine oxidases capable of detoxifying a fumonisin mycotoxin, including fumonisin mycotoxins bearing at least one tricarballylic ester substituent.

### BACKGROUND

Fumonisins are toxic secondary metabolites (mycotoxins) produced by various phytopathogenic fungi including several Fusarium and Aspergillus species. They predominantly contaminate corn and corn-based products and have also been detected on multiple grain-based products including oats, wheat, barley (C.F.I.A., 2017), as well as grapes (Qi et al., 2016; Renaud et al., 2015). Fumonisins are hepatotoxic and carcinogenic in animals (Gelderblom et al., 1991; Gelderblom et al., 1992; Voss et al., 2002) and cause equine leukoencephalomalacia (Marasas et al., 1988) and porcine pulmonary edema (Harrison et al., 1990). Consumption of fumonisin-contaminated food is correlated with neural tube defects (Missmer et al., 2006) and esophageal cancer in humans (Rheeder, 1992). As a result of climate change and modified agricultural practices, favorable conditions for fungal development and spread are expected to lead to an increase in fumonisin levels (Miller, 2001; Wu et al., 2011).

Fumonisins are a family of reduced linear polyketides that contain two tricarballylic ester groups and a primary amine derived typically from the condensation of L-alanine with the polyketide backbone. Fumonisin B1 (FB₁) is the most abundant fumonisin, while FB₂, FB₃, FB₄, and FB₆ chemotypes are also widespread and differ solely in the number and position of hydroxyl groups along the polyketide backbone. The chemical structure of fumonisin B2

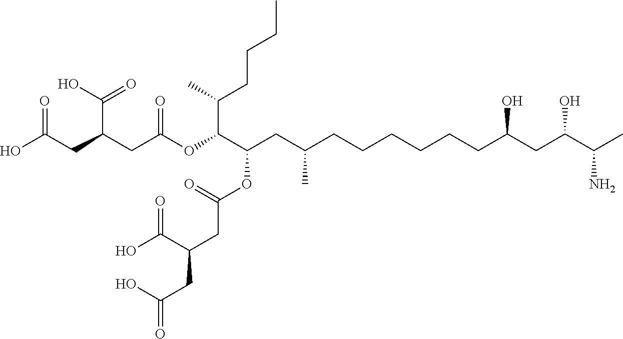 [SMILES: CCCC[C@@H](C)[C@@H](OC(=O)C[C@@H](CC(=O)O)C(=O)O)[C@H](C[C@@H](C)CCCCCC[C@@H](O)C[C@H](O)[C@H](C)N)OC(=O)C[C@@H](CC(=O)O)C(=O)O]

(FB₂) is provided above.

Fumonisins are structurally similar to sphingolipids and can act as competitive inhibitors of the enzyme ceramide synthase. The resulting sphingolipid imbalance upon inhibition endows fumonisins with their toxic and carcinogenic properties (Merrill et al., 2001; Riley et al., 2001). Both the tricarballylic ester and amine functional groups of fumonisins are thought to mediate toxicity with ceramide synthase. The amine group in particular is predicted to interact with the sphingoid-base binding site (Merrill et al., 2001; Norred et al., 2001).

Enzymatic modification of fumonisins is an attractive method to mitigate their toxicity (Vanhoutte et al., 2016). Fumonisin degrading enzymes have been identified in microorganisms that metabolize fumonisins as an energy source, but not in species that synthesize fumonisins. The bacteria Sphingomonas sp. ATCC 55552 (Duvick, 1998) and Sphingopyxis sp. MTA144 (Taubel, 2005) both contain a conserved gene cluster responsible for degrading fumonisins in a step-wise manner. Two gene products, FumD (carboxylesterase) and FumI (aminotransferase) within this cluster remove the fumonisin tricarballylic ester and amine functional groups respectively. Full de-esterification via FumD is required prior to deamination via FumI in order to render the fumonisin non-toxic (Hartinger et al., 2010; Hartinger et al., 2011; Heinl et al., 2011; Heinl et al., 2010). The carboxylesterase FumD is commercially available as FUMzyme® and is sold as a feed additive allowing for putative de-esterification within the animal's gut (Grenier et al., 2017).

The black yeast fungus Exophiala spinifera is also capable of metabolizing fumonisins as an energy source (Duvick, 1998; Duvick J., 2000; Duvick J., 1998). The gene cluster within Exophiala spinifera responsible for fumonisin degradation also produces a carboxylesterase that removes the tricarballylic ester moieties prior to oxidative deamination via an amine oxidase (Duvick, 1998; Duvick J., 2000; Duvick J., 1998). The wild-type amine oxidase requires hydrolyzed fumonisins as its substrate, however, subsequent engineered variants of the enzyme were capable of deaminating intact fumonisins (Chatterjee R., 2003).

Previously known wild-type enzymes isolated from native source (bacterial or fungal) that target the amine functional group of fumonisins require hydrolyzed fumonisins as substrates (ie: fumonisins lacking the tricarballylic ester moieties). This necessitates prior de-esterification via an additional enzyme that complicates the detoxification process. The aminotransferase FumI requires pyruvate as co-substrate and pyridoxal phosphate as co-enzyme (Hartinger et al., 2011). These requirements limit the usefulness of FumI as a fumonisin detoxification enzyme due to the expense of the cofactors and added complexity of the system.

It would be highly desirable to be provided with a means to detoxify products (such as, for example, corn-based and grain-based products) contaminated with fumonisins using a cost-effective one-step process. Seeing as current methods rely on enzymes that convert fumonisins into non-toxic metabolites following a sequential two-step process, namely, de-esterification followed by de-amination, and sometimes require expensive cofactors and co-substrates. A means that could simplify the fumonisin detoxification process would be beneficial.

### BRIEF SUMMARY

In one aspect, the present disclosure concerns a method of detoxifying a fumonisin mycotoxin, the method comprising treating the fumonisin mycotoxin with an enzyme isolated from a fumonisin-producing fungus, wherein the enzyme is active to catalyze oxidative deamination of the fumonisin mycotoxin, and wherein the fumonisin mycotoxin bears at least one tricarballylic ester substituent. In at least one embodiment, the fumonisin-producing fungus is a species of Aspergillus. In at least one embodiment, the enzyme is a recombinant enzyme.

According to a first aspect, the present disclosure provides a recombinant microbial host cell expressing an heterologous polypeptide having fumonisin amine oxidase activity. The recombinant microbial host cell comprises an heterologous nucleic acid molecule encoding the heterologous polypeptide having fumonisin amine oxidase activity, wherein the heterologous polypeptide has the amino acid sequence of SEQ ID NO: 5, SEQ ID NO: 27, SEQ ID NO: 28, or SEQ ID NO: 29, is a variant of the amino acid sequence of SEQ ID NO: 5, SEQ ID NO: 27, SEQ ID NO: 28 or SEQ ID NO: 29 having fumonisin amine oxidase activity or is a fragment of the amino acid sequence of SEQ ID NO: 5, SEQ ID NO: 27, SEQ ID NO: 28 or SEQ ID NO: 29 having fumonisin amine oxidase activity. In an embodiment, the variant or the fragment has at least 70%, 80%, 90% or 95% identity with respect to the amino acid sequence of SEQ ID NO: 5, SEQ ID NO: 27, SEQ ID NO: 28 or SEQ ID NO: 29. In another embodiment, the heterologous nucleic acid molecule allows the expression of an intracellular form of the heterologous polypeptide having fumonisin amine oxidase activity. In a further embodiment, the heterologous nucleic acid molecule allows the expression of a secreted form of the heterologous polypeptide having fumonisin amine oxidase activity. In still a further embodiment, the heterologous nucleic acid molecule is operatively associated with a further nucleic acid molecule encoding a signal sequence peptide. In another embodiment, the heterologous nucleic acid molecule allows the expression of a membrane-associated form of the polypeptide having heterologous fumonisin amine oxidase activity. In a specific embodiment, the membrane-associated form of the heterologous polypeptide having fumonisin amine oxidase activity is a tethered form of the heterologous polypeptide having fumonisin amine oxidase activity. In an embodiment, the recombinant microbial host cell is a yeast host cell. The recombinant microbial host can be from the genus Saccharomyces and, in a some additional embodiments, from the species Saccharomyces cerevisiae. The recombinant microbial host cell can be from the genus Pichia and, in some additional embodiments, from the species Pichia pastoris. In an embodiment, the recombinant microbial host cell can be a fungal host cell. The recombinant microbial host cell can be from the genus Aspergillus or Trichoderma. The recombinant microbial host can be a bacterial host cell. The recombinant microbial host cell can be from the genus Bacillus, and in some additional embodiments, from the species Bacillus subtilis. The recombinant microbial host cell can be from the genus Escherichia, and in some additional embodiments, from the species Escherichia coli.

According to a second aspect, the present disclosure provides a microbial composition comprising (i) the heterologous polypeptide having fumonisin amine oxidase activity described herein and (ii) the recombinant microbial host cell described or at least one component from the recombinant microbial host cell described herein. In an embodiment, the microbial composition comprises the recombinant microbial host cell. In another embodiment, the microbial composition comprises the at least one component from the recombinant microbial host cell. In an embodiment, the at least one component comprises or is from a lysed recombinant microbial host cell.

According to a third aspect, the present disclosure provides a process for making an isolated, synthetic or recombinant polypeptide having heterologous fumonisin amine oxidase activity. The process comprises a) propagating the recombinant microbial host cell described herein to obtain a propagated recombinant microbial host cell and the heterologous fumonisin amine oxidase; b) dissociating the propagated microbial host cell from the heterologous polypeptide having fumonisin amine oxidase activity to obtain a dissociated fraction enriched in the heterologous polypeptide having the fumonisin amine oxidase activity or lysing the propagated microbial host cell to obtained a lysed fraction; c) optionally drying the dissociated or lysed microbial host cell to obtain a dried fraction; and d) substantially purifying the heterologous polypeptide having fumonisin amine oxidase activity from the dissociated, lysed or dried fraction to provide the isolated, synthetic or recombinant heterologous polypeptide having fumonisin amine oxidase activity.

According to a fourth aspect, the present disclosure provides a process for making a microbial composition comprising the recombinant polypeptide having heterologous fumonisin amine oxidase activity described herein. The process comprises a) propagating the recombinant microbial host cell described herein to obtain a propagated recombinant microbial host cell and the heterologous fumonisin amine oxidase; and b) formulating the propagated microbial host cells into the microbial composition. Optionally, the process can comprise optionally enriching the composition with the propagated microbial host cell (by filtration for example), drying and/or freezing the microbial composition.

According to a fifth aspect, the present disclosure provides a process for making a microbial product comprising the recombinant polypeptide having heterologous fumonisin amine oxidase activity described herein. The process comprises a) propagating the recombinant microbial host cell described herein to obtain a propagated recombinant microbial host cell and the heterologous fumonisin amine oxidase or being provided with propagated recombinant microbial host cells; b) dissociating the propagated microbial host cell from the heterologous polypeptide having fumonisin amine oxidase activity to obtain a dissociated fraction enriched in the heterologous polypeptide having the fumonisin amine oxidase activity or lysing the propagated microbial host cell to obtained a lysed fraction; c) optionally drying the dissociated or lysed microbial host cell to obtain a dried fraction; and d) optionally substantially purifying the heterologous polypeptide having fumonisin amine oxidase activity from the dissociated, lysed or dried fraction to provide the isolated, synthetic or recombinant heterologous polypeptide having fumonisin amine oxidase activity.

According to a sixth aspect, the present disclosure provides an isolated, synthetic or recombinant polypeptide having the amino acid sequence of SEQ ID NO: 5, SEQ ID NO: 27, SEQ ID NO: 28 or SEQ ID NO: 29 being a variant of the amino acid sequence of SEQ ID NO: 5, SEQ ID NO: 27, SEQ ID NO: 28 or SEQ ID NO: 29 having fumonisin amine oxidase activity or a fragment of the amino acid sequence of SEQ ID NO: 5, SEQ ID NO: 27, SEQ ID NO: 28 or SEQ ID NO: 29 having fumonisin amine oxidase activity. In an embodiment, the isolated, synthetic or recombinant polypeptide of claim 27, wherein the variant or the fragment has at least 70%, 80%, 90% or 95% identity with respect to the amino acid sequence of SEQ ID NO: 5, SEQ ID NO: 27, SEQ ID NO: 28 or SEQ ID NO: 29.

According to a seventh aspect, the present disclosure provides a method for detoxifying a fumonisin mycotoxin. The method comprises contacting the microbial recombinant yeast host cell described herein, the microbial composition described herein, or the isolated, synthetic or recombinant polypeptide described herein with the fumonisin mycotoxin so as to cause the deamination of the fumonisin mycotoxin into an oxidized fumonisin mycotoxin. In an embodiment, the fumonisin mycotoxin bears at least one tricarballylic ester substituent. In another embodiment, the method is for making a feed product. In a further embodiment, the feed product is or comprises silage, hay, straw, grains, grain by-products, legumes, cottonseed meal, vegetables, milk and/or milk by-products. In another embodiment, the feed is or comprises grain by-products. In a further embodiment, the grain by-products are distillers grains. In another embodiment, the method is for making a food product. In still another embodiment, the food product is or comprises a flour, such as, for example, corn flour.

According to an eighth aspect, the present disclosure provides a feed product comprising the isolated, synthetic or recombinant polypeptide described herein. In an embodiment, the feed product further comprises the recombinant microbial host cell described herein or at least one component from the recombinant microbial host cell described herein. The feed product can be or comprise silage, hay, straw, grains, grain by-products, legumes, cottonseed meal, vegetables, milk and/or milk by-products. In an embodiment, the feed can be or comprise grain by-products. In still a further embodiment, the grain by-products are distillers grains. In yet a further embodiment, the feed product further comprises an additive, such as, for example, a yeast cell wall, a binder or a further mycotoxin-degrading enzyme.

According to a seventh aspect, the present disclosure provides a food product comprising the isolated, synthetic or recombinant polypeptide described herein. In an embodiment, the food product further comprises the recombinant microbial host cell described herein or at least one component from the recombinant microbial host cell described herein. In still a further embodiment, the food product is or comprises a flour, such as, for example, corn flour.

### BRIEF DESCRIPTION OF THE DRAWINGS

FIG. 1 shows an embodiment of a purification scheme to enrich for fumonisin deamination activity from culture supernatants of Aspergillus niger.

FIG. 2 shows a representative reverse-phase liquid chromatography-mass spectrometry (LC-MS) spectrum of FB₂ conversion into FPy₂ via incubation with activity-enriched samples. Both species were monitored via their signal intensity upon detection in the Orbitrap™ mass spectrometer. FPy₂ is 1.03 Da lighter than FB₂ and typically elutes 0.46 minutes later in the separation method described in the examples.

FIG. 3 shows a chromatographic enrichment of fumonisin deamination activity from A. niger culture supernatants. The photograph on the left depicts the visual appearance of the Q-Sepharose™ column before and after sample application. The photograph on the right highlights the appearance of the sample after a 50 mM NaCl wash (fraction 1) and the eluates obtained following the addition of 250 mM (fraction 2), 500 mM (fraction 3), and 1000 mM NaCl (fraction 4). Batch elution of deamination activity at 250 mM NaCl off the Q-Sepharose™ anion exchange column removes contaminating pigment and nucleic acids.

FIG. 4 shows the elution profile following Phenyl-Sepharose™ enrichment of deamination activity. Grey bars represent deamination activity (measured as % conversion of intact FB₂ to FPy₂, right axis) of individual fractions as monitored via reverse phase LC-MS. Solid line represents absorbance at 280 nm (left axis). Dashed black line represents conductivity (mS/cm). Horizontal lines (i) and (ii) delineate the samples that were pooled together for subsequent analysis.

FIGS. 5A and 5B show gel permeation chromatograms of active pooled samples (i) and (ii) following Phenyl-Sepharose™ enrichment. Grey bars represent deamination activity (measured as % conversion of intact FB₂ to FPy₂, right axis) of individual fractions as monitored via reverse phase LC-MS. Solid line represents absorbance at 280 nm (left axis).

FIGS. 6A and 6B show high-resolution mono-Q™ anion exchange chromatograms of active samples pooled following gel permeation chromatography. Grey bars represent deamination activity (measured as % conversion of intact FB₂ to FPy₂, right axis) of individual fractions as monitored via reverse phase LC-MS. Solid line represents absorbance at 280 nm (left axis). Dashed black line represents conductivity (mS/cm). Peaks labelled I-V were subjected to proteomics analysis to identify candidate fumonisin deamination enzymes.

FIG. 7 shows the temperature dependence of deamination activity. Results are shown as the relative activity (in %) as a function of temperature (in ° C.). Error bars represent standard error of the mean (n=2).

FIG. 8 shows the pH dependence of deamination activity. Results are shown as the relative activity (in %) as a function of pH. Error bars represent standard error of the mean (n=2).

FIG. 9 shows fumonisin chemotype preference of deamination activity. Error bars represent standard error of the mean (n=2). Dashed lines represent initial conversion rates of intact to deaminated fumonisins (FB₂ to FPy₂=24.3±1.5% hr⁻¹ represented by solid lines and dark circles; FB₁ to FPy₁=2.1±0.1% hr⁻¹ represented by a dashed line and open circles).

FIG. 10 shows the reverse-phase LC-MS/MS peptide spectrum of the native amine oxidase residues 194-206. The sequence derived from the y-series ions is listed above the spectrum. Accurate detection of the b₂, b₃, and b₄ ions allowed for complete sequencing of the entire tryptic peptide.

FIGS. 11A to 11D show reverse-phase LC/MS analysis of FB₂ incubated in the presence or absence of 6 nM homogenous recombinant AnFAO for 1 hour at 37° C. (FIGS. 11A and 11B) Reverse-phase LC/MS analysis of FB₂ incubated in the presence of enzyme for 1 hour at 37° C. (FIG. 11A) The relative abundance (%) over elution time (min) for FPy₂ which elutes distinctly later (3.38 minutes) than FB₂. Panel A inset shows a Coomassie-stained SDS-PAGE analysis of purified recombinant AnFAO post gel permeation chromatography. PM=protein markers. Numbers represent MW of standards in kDa. (FIG. 11B) The relative abundance (%) over mass (Da) for FPy₂ which has an [M-H]⁻ of 703.3563. (FIGS. 11C and 11D) Reverse-phase LC/MS analysis of FB₂ incubated in the absence of enzyme for 1 hour at 37° C. (FIG. 11C) The relative abundance (%) over elution time (min) for intact FB₂ which elutes at 2.92 minutes. (FIG. 11D) The relative abundance (%) as a function of mass (Da) for intact FB₂ which has an [M-H]⁻ of 704.3828.

FIG. 12A to 12D demonstrate that Pichia pastoris produces active recombinant AnFAO and that recombinant AnFAO is a non-covalent flavoprotein. (FIG. 12A) The FB₂ deamination activity (% conversion) obtained from culture supernatants (culture sup.), live cells, and cell lysates tested 6 and/or 24 hours post-induction with methanol. Both secreted (pPICZαA-FAO) and intracellular (pPICZB-FAO) recombinant proteins can deaminate FB₂ following methanol-induced expression. Insets represent western blots probing for the 6× His-tagged recombinant secreted or intracellular AnFAO. (FIG. 12B) The absorbance obtained at different wavelengths (nm) of AnFAO_15309 following exhaustive dialysis against 20 mM MES (pH 6), 150 mM NaCl, and either in the presence (solid line) or absence (dashed line) of 10 μM Flavin Adenine Dinucleotide (FAD). (FIG. 12C) The relative enzyme rate (%) as a function of AnFAO in the presence or absence of excess FAD. Error bars represent standard deviation (n=3). (FIG. 12D) A Q-Exactive Orbitrap high resolution whole mass spectrum of AnFAO showing the peak intensity on the y-axis over mass (Da) obtained following separation on an Agilent 1290 ultra-high-performance liquid chromatography system equipped with a ZORBAX RRHD C18 column (100×2.1 mm, 1.8 mm, RRHD C18 column, 300 particle size). In particular, the column was maintained at 80° C. with a 300 μL/min flow rate. Mobile phase A (H2O, 0.1% FA) was held at 95% for 30 s and mobile phase B (acetonitrile 0.1% FA) was increased linearly to 100% over 8 min (Irvine et al., 2017).

FIG. 13 shows relative activity rates of AnFAO clones 15309, 6142, 10927, and 7097 as determined via Amplex™ red assay. Results are shown as the relative activity (in %) as a function of the clone used. Error bars represent standard deviation (n=3).

FIG. 14. Relative activity rates of AnFAO_15309 towards non-fumonisin substrates as determined via Amplex™ red assay. All rates set relative to FB₃. Results are shown as the relative activity (in %) as a function of the substrate used. Error bars represent standard deviation (n=3).

FIGS. 15A and 15B show the relative fumonisin deamination rates of AnFAO_15309 when held at the indicated temperatures. (FIG. 15A) Results are shown as the relative activity (in %) as a function of temperature (° C.). Error bars represent standard error of the mean (n=3). (FIG. 15B) Circular dichroism thermal denaturation (melting) curves of AnFAO clones 15309 (thin line), 6142 (dashed line), and 10927 (thick line). Results are shown as the mean residue ellipticity as a function of temperature (° C.).

FIGS. 16A to 16C show (FIG. 16A) pH dependence, (FIG. 16B) NaCl dependence, and (FIG. 16C) ethanol tolerance of AnFAO_15309. Results are shown as the relative activity (in %) as a function of pH (A), NaCl concentration (mM) (B) and volume percentage of ethanol (C). Error bars represent standard deviation (n=3) for all experiments.

FIGS. 17A and 17B show fumonisin deamination activity of AnFAO_15309. (FIG. 17A) Absorbance (571 nm) vs. time (minutes) plot for AnFAO (80 nM) in the presence of 25 μM FB₁. (FIG. 17B) Absorbance (571 nm) vs. time (minutes) plot for GST-tagged reactive intermediate deaminase plus amine oxidase (RID+AO) enzyme in the presence of 25 μM FB₁. Inset represents SDS-PAGE analysis of purified GST-tagged RID+AO protein. Error bars represent standard error of the mean (n=3) for all time points. PM=protein markers. Numbers down the left side represent molecular weight markers (kDa).

FIGS. 18A to 18C illustrate that AnFAO can be functionally expressed in Saccharomyces cerevisiae. (FIG. 18A) Graphic representation of the AnFAO expression cassette integrated into S. cerevisiae. (FIG. 18B) Western blot analysis of S. cerevisiae codon-optimized AnFAO probed for a C-terminal His-tag shows a prominent band of AnFAO at the predicted MW. AnFAO was expressed using a copy of the native Saccharomyces cerevisiae promoter from the TEF2 gene and terminator from the ADH3 gene. (FIG. 18C) The LC-MS peak area (Millions) for soluble fractions of the AnFAO expressing strain (indicated as “=AnFAO)) or wild type (indicated as “wt)). FB₁ and FB₂ were deaminated only when AnFAO was expressed.

FIGS. 19A to 19C show the reverse phase LC-MS analysis of corn quality control material from Romer Labs contaminated with 667±78 FB₁, 156±21 FB₂, and 89±22 FB₃. A) LC-MS analysis monitoring for FB₁ and FPy₁. B) LC-MS analysis monitoring for FB₂ and FPy₂. C) LC-MS analysis monitoring for FB₃ and FPy₃. Solid lines represent samples at 0 h, while dashed lines represent samples after 16 hours treatment with AnFAO. No intact fumonisin remained following AnFAO treatment.

Having thus generally described the nature of the invention, reference will now be made to the accompanying drawings, showing by way of illustration, a preferred embodiment thereof, and in which:

### DETAILED DESCRIPTION

Novel enzymatic activity has been identified in culture supernatants of fumonisin-producing Aspergillus strains. This enzymatic activity is capable of replacing the amine functional group of a fumonisin (for example, FB₂, below) with an oxo group to produce an oxidized fumonisin (for example, FPy₂, below) (Burgess et al., 2016; Qi et al., 2016; Renaud et al., 2015).

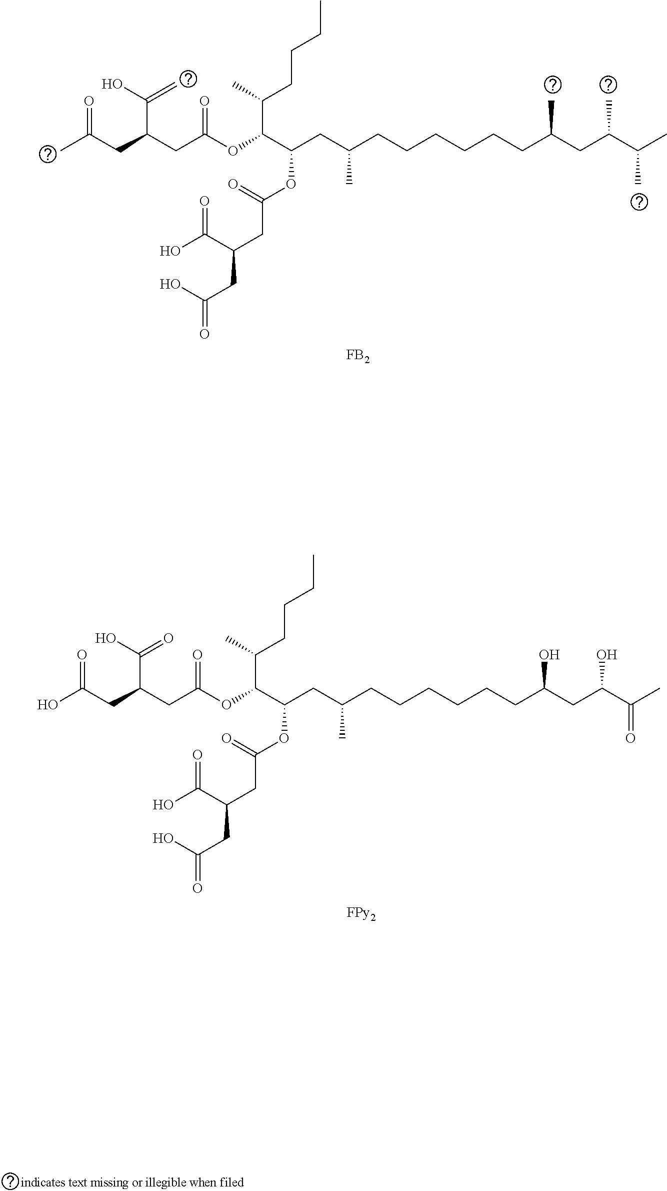

The oxidized fumonisins are an order of magnitude less toxic than the intact fumonisins, as determined using a duckweed (Lemna minor) plant growth assay (Burgess et al., 2016). A protocol has been developed to enrich for this deamination activity from the fungal source.

An active recombinant version of the newly identified enzyme has also been produced in the heterologous hosts Escherichia coli, Pichia pastoris, and Saccharomyces cerevisiae. It is envisioned that this enzyme might be leveraged as a tool to reduce the toxicity of fumonisins in contaminated feed samples, either as a pure enzymatic preparation or via the engineering of a microbe bearing the enzyme enabling in situ fumonisin detoxification.

Heterologous Fumonisin Amine Oxidases

The present disclosure relates to polypeptides having fumonisin amine oxidase activity to allow the detoxification of fumonisins, especially fumonisins bearing at least one or two tricarballylic ester substituents. The use of such polypeptides, in some embodiments, reduces the complexity in the detoxification process because a single polypeptide exhibiting fumonisin oxidase activity is sufficient to reduce the toxicity of the fumonisin. The polypeptides having fumonisin amine oxidase activity of the present disclosure are intended to be expressed in a recombinant microbial host cell. The polypeptides can be provided from a recombinant microbial host cell or a composition or a product from the recombinant microbial host cell.

The polypeptides of the present disclosure have fumonisin amine oxidase activity and are monoamine oxidases. Polypeptides having monoamine oxidase activity (EC 1.4.3.4) catalyze the oxidation of amine-containing compounds into their corresponding imines, which then hydrolyze non-enzymatically to their respective aldehydes or ketones. Monoamine oxidases require flavin adenine dinucleotide (FAD) as a cofactor. Polypeptides having fumonisin amine oxidase activity include, as a substrate, fumonisin. In some embodiments, polypeptides having fumonisin amine oxidase activity include, as a substrate, a fumonisin bearing at least one tricarballylic ester substituent.

The polypeptide having fumonisin oxidase activity can be derived from an organism which also produces the fumonisin toxin, such as, for example Aspergillus niger. In an embodiment, the polypeptide having fumonisin oxidase activity comprises or consists essentially of the amino acid sequence of SEQ ID NO: 5, 27, 28 or 29. In a specific embodiment, the polypeptide having fumonisin oxidase activity comprises of the amino acid sequence of SEQ ID NO: 5. In a specific embodiment, the polypeptide having fumonisin oxidase activity consists essentially of the amino acid sequence of SEQ ID NO: 5. In a specific embodiment, the polypeptide having fumonisin oxidase activity comprises of the amino acid sequence of SEQ ID NO: 27. In a specific embodiment, the polypeptide having fumonisin oxidase activity consists essentially of the amino acid sequence of SEQ ID NO: 27. In a specific embodiment, the polypeptide having fumonisin oxidase activity comprises of the amino acid sequence of SEQ ID NO: 28. In a specific embodiment, the polypeptide having fumonisin oxidase activity consists essentially of the amino acid sequence of SEQ ID NO: 28. In a specific embodiment, the polypeptide having fumonisin oxidase activity comprises of the amino acid sequence of SEQ ID NO: 29. In a specific embodiment, the polypeptide having fumonisin oxidase activity consists essentially of the amino acid sequence of SEQ ID NO: 29. In the context of the present disclosure, a polypeptide having fumonisin oxidase activity consisting essentially of the amino acid sequence of SEQ ID NO: 5, 27, 28, or 29 can include additional amino acid residues at the amino or carboxyl end of the polypeptide, provided that these additional amino acid residues do not alter the fumonisin amine oxidase activity of the polypeptide. In some embodiments, the polypeptide having fumonisin oxidase activity consisting essentially of the amino acid sequence of SEQ ID NO: 5, 27, 28 or 29 includes at least one, two, three, four, five, six, seven, eight, nine or ten additional amino acid residues at the amino and/or the carboxyl end of the polypeptide, provided that these additional amino acid residues do not alter the fumonisin amine oxidase activity of the polypeptide.

In the context of the present disclosure, a polypeptide having fumonisin amine oxidase activity means that the polypeptides exhibit relative fumonisin amine oxidase activity of at least 10%, 20%, 30%, 40%, 50%, 60%, 70%, 80%, 90%, 95%, 96%, 97%, 98% or 99% of the fumonisin amine oxidase activity of SEQ ID NO: 5, 27, 28 or 29. The fumonisin amine oxidase activity of a polypeptide is determined in the presence of its cofactor (FAD), at different temperatures (e.g., between 4 and 95° C., and, in some embodiments, at 37° C.) as well as at different pH (e.g., between 3 to 8 and, in some embodiments, at pH 6). Assays for determining fumonisin amine oxidase activity include, without limitation, spectrophotometric and chromatography (e.g., high performance liquid chromatography) assays.

In an embodiment, the polypeptide having fumonisin amine oxidase activity is a variant and/or a fragment of the amino acid sequence of SEQ ID NO: 5, 27, 28 or 29. In a specific embodiment, the polypeptide having fumonisin amine oxidase activity is a variant and/or a fragment of SEQ ID NO: 5. In a specific embodiment, the polypeptide having fumonisin amine oxidase activity is a variant and/or a fragment of SEQ ID NO: 27. In a specific embodiment, the polypeptide having fumonisin amine oxidase activity is a variant and/or a fragment of SEQ ID NO: 28. In a specific embodiment, the polypeptide having fumonisin amine oxidase activity is a variant and/or a fragment of SEQ ID NO: 29. A variant comprises at least one amino acid difference (substitution or addition) when compared to the amino acid sequence of SEQ ID NO: 5, 27, 28 or 29. A fragment comprises at least one less amino acid residue (deletion) than the amino acid sequence of SEQ ID NO: 5, 27, 28 or 29. A fragment of a variant of the amino acid sequence of SEQ ID NO: 5 comprises at least one amino acid difference and at least one amino acid residue deletion (when compared to the amino acid sequence of SEQ ID NO: 5, 27, 28 or 29). The variants and fragments of the present disclosure exhibit fumonisin amine oxidase activity. In an embodiment, the variants or fragments exhibits at least 50%, 60%, 70%, 80%, 90%, 95%, 96%, 97%, 98% or 99% of the activity of the wild-type fumonisin amine oxidase polypeptide having the amino acid sequence of SEQ ID NO: 5, 27, 28 or 29. In some embodiments, the variants and the fragments can also have at least 70%, 80%, 85%, 90%, 95%, 96%, 97%, 98% or 99% identity to the amino acid sequence of SEQ ID NO: 5, 27, 28 or 29. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant fumonisin amine oxidase polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant fumonisin amine oxidase polypeptide can also be a conservative variant or an allelic variant. As used herein, a conservative variant refers to alterations in the amino acid sequence that do not adversely affect the biological functions of the fumonisin amine oxidase (e.g., detoxification of fumonisin). A substitution, insertion or deletion is said to adversely affect the polypeptide when the altered sequence prevents or disrupts a biological function associated with the polypeptide (e.g., the oxidation of a fumonisin substrate). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the fumonisin amine oxidase.

In the context of the present disclosure, the intracellularly expressed heterologous polypeptide can be modified at the N-terminus to provide variant or fragment heterologous polypeptides. If the heterologous polypeptide includes a native signal sequence, it can be removed to allow the intracellular expression of the heterologous polypeptide, variant or fragment. As such, the heterologous polypeptide, variant or fragment can lack any signal sequence. In some embodiments, the intracellularly expressed heterologous polypeptide is selected to have or is modified to have a first methionine residue (e.g., a methionine residue at position 1). In some embodiments, 1, 2, 3, 4, 5, 6, 7, 8, 9, 10, or more consecutive amino acid residues are removed from the native sequence and optionally at the N-terminus, after the first methionine. In some embodiments, the removed amino acid residues can be positioned right next (e.g., following) to the first methionine. Alternatively or in combination, 1, 2, 3, 4, 5, 6, 7, 8, 9, 10, or more consecutive amino acid residues are added starting at the second position from the N-terminus, following the first methionine. In some embodiments, 1, 2, 3, 4, 5, 6, 7, 8, 9, 10, or more amino acid residues are removed starting at the second position from the N-terminus, following the first methionine. In some embodiments, both 1, 2, 3, 4, 5, 6, 7, 8, 9, 10, or more amino acid residues are removed and 1, 2, 3, 4, 5, 6, 7, 8, 9, 10, or more amino acid residues are added, starting at the second position from the N-terminus, following the first methionine. In some specific embodiments, a single amino acid residue (e.g., at position 2) is removed, following the first methionine. In such embodiment, one or more consecutive amino acid residues can be added at the site of the deletion. In some alternative embodiments, two consecutive amino acid residues (e.g., at positions 2 and 3) are removed, following the first methionine. In such embodiment, one, two or more consecutive amino acid residues can be added at the site of the deletion. In some additional embodiments, three consecutive amino acid residues (e.g., at positions 2 to 4) are removed, following the first methionine. In such embodiment, one, two or three consecutive amino acid residues can be added at the site of the deletion. In some further embodiments, four consecutive amino acid residues (e.g., at positions 2 to 5) are removed following the first methionine. In some embodiments, one, two, three or four consecutive amino acid residues are added at the site of the deletion.

In some embodiments, a fragment polypeptide can correspond to the fumonisin amine oxidase described herein to which the signal peptide sequence has been removed. In other embodiments, the fragment can be, for example, a truncation of one or more amino acid residues at the amino-terminus, the carboxy terminus or both terminus of the polypeptide having fumonisin amine oxidase activity or variant. Alternatively or in combination, the fragment can be generated from removing one or more internal amino acid residues. In an embodiment, the fragment of the polypeptide having fumonisin amine oxidase activity has at least 100, 150, 200, 250, 300, 350, 400 or more consecutive amino acids of the fumonisin amine oxidase or the variant.

In an embodiment, the polypeptide having fumonisin amine oxidase activity includes variants and fragments having, at a position corresponding to location 445 of the amino acid sequence of SEQ ID NO: 5, a glycine residue. In another embodiment, the polypeptide having fumonisin amine oxidase activity does not include (e.g., excludes) variants having, at position 445, a residue other than a glycine residue, such as, for example, a glutamic acid residue.

The polypeptide of the present disclosure can be designed to be expressed for secretion outside the recombinant yeast host cell. In some embodiments, the polypeptide includes one or a combination of signal peptide sequence(s) allowing the transport of the polypeptide outside the yeast host cell's wall (e.g., in a secreted form). The signal sequence can simply be added to the polypeptide or replace the signal peptide sequence already present in the polypeptide from which the fumonisin amine oxidase portion is derived. The signal sequence can be native or heterologous to the protein from which the fumonisin amine oxidase portion is derived. In some embodiments, one or more signal sequences can be used. It is understood that the one or more signal sequences are cleaved once the heterologous polypeptide is secreted. In some embodiments, the signal sequence is from the invertase protein (and can have, for example, the amino acid sequence of SEQ ID NO: 7, be a variant of the amino acid sequence of SEQ ID NO: 7 or be a fragment of the amino acid sequence of SEQ ID NO: 7); the AGA2 protein (and can have, for example, the amino acid sequence of SEQ ID NO: 7, be a variant of the amino acid sequence of SEQ ID NO: 7 or be a fragment of the amino acid sequence of SEQ ID NO: 7); or the α-mating factor protein (and can have, for example, the amino acid sequence of SEQ ID NO: 9, be a variant of the amino acid sequence of SEQ ID NO: 9 or be a fragment of the amino acid sequence of SEQ ID NO: 9).

In the context of the present disclosure, the expression “functional variant of a signal sequence” refers to an amino acid sequence that has been substituted in at least one amino acid position when compared to the native signal sequence and which retain the ability to direct the expression of the polypeptide outside the cell, in a secreted form. In the context of the present disclosure, the expression “functional fragment of a signal sequence” refers to a shorter amino acid sequence than the native signal sequence that retains the ability to direct the expression of the polypeptide outside the cell.

Recombinant Host Cells

The polypeptides described herein can independently be provided in an isolated, synthetic or recombinant form (derived from the recombinant microbial host cell described herein) or derived from a recombinant microbial host cell expressing the heterologous polypeptide. The recombinant microbial cell thus includes at least one genetic modification. In the context of the present disclosure, when recombinant microbial cell is qualified as “having a genetic modification” or as being “genetically engineered”, it is understood to mean that it has been manipulated to either add at least one or more heterologous or exogenous nucleic acid residue and/or remove at least one endogenous (or native) nucleic acid residue. The genetic manipulations did not occur in nature and are the results of in vitro manipulations of the recombinant host cell. When the genetic modification is the addition of an heterologous nucleic acid molecule, such addition can be made once or multiple times at the same or different integration sites. When the genetic modification is the modification of an endogenous nucleic acid molecule, it can be made in one or both copies of the targeted gene.

When expressed in a recombinant microbial host cell, the heterologous polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the recombinant microbial host cell. “Heterologous” also includes a native coding region, or portion thereof, that is introduced into the source organism in a form that is different from the corresponding native gene, e.g., not in its natural location in the organism's genome. The heterologous nucleic acid molecule is purposively introduced into the recombinant host cell.

Thus, for example, an heterologous element could be derived from a different strain of host cell, or from an organism of a different taxonomic group (e.g., different domain, kingdom, phylum, class, order, family, genus, or species, or any subgroup within one of these classifications).

When an heterologous nucleic acid molecule is present in the recombinant microbial host cell, it can be integrated in the host cell's genome. The term “integrated” as used herein refers to genetic elements that are placed, through molecular biology techniques, into the genome of a microbial host cell. For example, genetic elements can be placed into the chromosomes of the microbial host cell as opposed to in a vector such as a plasmid carried by the host cell. Methods for integrating genetic elements into the genome of a host cell are well known in the art and include homologous recombination. The heterologous nucleic acid molecule can be present in one or more copies in the microbial host cell's genome. For example, the heterologous nucleic acid molecule can be present in 1, 2, 3, 4, 5, 6, 7, 8 or more copies in the microbial host cell's genome. Alternatively, the heterologous nucleic acid molecule can be independently replicating from the microbes' genome. In such embodiment, the nucleic acid molecule can be stable and self-replicating.

In the context of the present disclosure, a “microbial host cell” can be a bacterial host cell, a yeast host cell or a fungal host cell. The term “microbial host cell” necessarily excludes animal (including mammalian) and insect cells.

In the context of the present disclosure, the recombinant host cell can be a recombinant fungal cell, such as, for example, a recombinant yeast host cell or a recombinant mold host cell. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, S. boulardfi, K. lactis, K. marxianus or K. fragilis. In some embodiments, the recombinant yeast host cell is selected from the group consisting of Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenfi, Debaryomyces polymorphus, Schizosaccharomyces pombe and Schwanniomyces occidentalis. In some additional embodiments, the recombinant yeast host cell is from Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenfi, Debaryomyces polymorphus, Schizosaccharomyces pombe and/or Schwanniomyces occidentalis. In some embodiments, the recombinant host cell can be an oleaginous yeast cell. For example, the recombinant oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant host cell can be an oleaginous microalgae host cell (e.g., for example, from the genera Thraustochytrium or Schizochytrium). In an embodiment, the recombinant yeast host cell is from the genus Saccharomyces and, in some embodiments, from the species Saccharomyces cerevisiae. In an embodiment, the recombinant yeast host cell is from the genus Pichia and, in some embodiments, from the species Pichia pastoris.

Suitable fungal host cell can be, for example, from the genus Aspergillus or Trichoderma.

Polypeptides having the fumonisin amine oxidase activity are expressed from one or more heterologous nucleic acid molecules in one or more recombinant microbial host cell. As such, the polypeptide having fumonisin oxidase activity are heterologous with respect to the recombinant microbial host cell expressing them. As used herein, the term “heterologous” when used in reference to a nucleic acid molecule (such as a promoter, a terminator or a coding sequence) or a polypeptide refers to a nucleic acid molecule or a polypeptide that is not natively found in the recombinant host cell. “Heterologous” also includes a native coding region/promoter/terminator, or portion thereof, that was introduced into the source organism in a form and/or at a location that is different from the corresponding native gene, e.g., not in its natural location in the organism's genome. The heterologous nucleic acid molecule is purposively introduced into the recombinant microbial host cell. For example, a heterologous element could be derived from a different strain of host cell, or from an organism of a different taxonomic group (e.g., different domain, kingdom, phylum, class, order, family, genus, or species, or any subgroup within one of these classifications).

The microbial host cell can be a bacterial host cell. Suitable bacterial host cells that can be genetically modified as described herein can be a Gram-positive or a Gram-negative bacteria. The recombinant bacterial host cell can be, for example, from the phylum Acidobacteria, Actinobacteria, Aquificae, Bacteroidetes, Chlamydiae, Cholorobi, Chloroflexi, Chrysiogenetes, Cyanobacteria, Deferribacteres, Deinococcus-Thermus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermotogae or Verrucomicrobia. In some embodiments, the bacterial host cell is from one of the following genus Acidobacterium, Geothrix, Holophaga, Acidimicrobium. Kribella, Atopobium, Collinsella, Coriobacterium, Cryptobacterium, Denitrobacterium, Eggerthella, Slackia, Rubrobacter, Sphaerobacter, Aquifex, Hydrogenivirga, Hydrogenobacter, Hydrogenobaculum, Thermocrinis, Hydrogenothermus, Persephonella, Sulfurihydrogenibium, Venenivibrio, Bacteroides, Acetofilamentum, Acetomicrobium, Acetothermus, Anaerorhabdus, Megamonas, Rikenella, Marinilabilia, Porphyromonas, Dysgonomonas, Prevotella, Chlamydia, Chlamydophila, Simkania, Fritschea, Simkania, Fritschea, Chrysiogenes, Deferribacter, Denitrovibrio, Flexistipes, Geovibrio, Deinococcus, Thermus, Meiothermus, Marinithermus, Oceanithermus, Vulcanithermus, Dictyoglomus, Hepatoplasma, Mycoplasma, Ureaplasma, Entomoplasma, Mesoplasma, Spiroplasma, Anaeroplasma, Asteroleplasma, Erysipelothrix, Holdemania, Acholeplasma, Phytoplasma, Fusobacterium, Gemmatimonas, Nitrospira, Gemmata, Isosphaera, Pirellula, Planctomyces, Brocadia, Kuenenia, Scalindua, Anammoxoglobus, Jettenia, Asticcacaulis, Brevundimonas, Caulobacter, Phenylobacterium, Kordiimonas, Parvularcula, Aurantimonas, Fulvimarina, Bartonella, Beijerinckia, Chelatococcus, Derxia, Methylocella, Afipia, Agromonas, Blastobacter, Bosea, Bradyrhizobium, Nitrobacter, Oligotropha, Photorhizobium, Rhodoblastus, Rhodopseudomonas, Brucella, Mycoplana, Ochrobactrum, Ancalomicrobium, Ancylobacter, Angulomicrobium, Aquabacter, Azorhizobium, Blastochloris, Devosia, Dichotomicrobium, Filomicrobium, Gemmiger, Hyphomicrobium, Labrys, Methylorhabdus, Pedomicrobium, Prosthecomicrobium, Rhodomicrobium, Rhodoplanes, Seliberia, Starkeya, Xanthobacter, Methylobacterium, Microvirga, Protomonas, Roseomonas, Methylocystis, Methylosinus, Methylopila, Aminobacter, Aquamicrobium, Defluvibacter, Hoeflea, Mesorhizobium, Nitratireductor, Parvibaculum, Phyllobacterium, Pseudaminobacter, Agrobacterium, Rhizobium, Sinorhizobium, Liberibacter, Ahrensia, Albidovulum, Amaricoccus, Antarctobacter, Catellibacterium, Citreicella, Dinoroseobacter, Haematobacter, Jannaschia, Ketogulonicigenium, Leisingera, Loktanella, Maribius, Marinosulfonomonas, Marinovum, Maritimibacter, Methylarcula, Nereida, Oceanibulbus, Oceanicola, Octadecabacter, Palleronia, Pannonibacter, Paracoccus, Phaeobacter, Pseudorhodobacter, Pseudovibrio, Rhodobaca, Rhodobacter, Rhodothalassium, Rhodovulum, Roseibacterium, Roseibium, Roseicyclus, Roseinatronobacter, Roseisalinus, Roseivivax, Roseobacter, Roseovarius, Rubrimonas, Ruegeria, Sagittula, Salipiger, Silicibacter, Staleya, Stappia, Sulfitobacter, Tetracoccus, Thalassobacter, Thalassobius, Thioclava, Yangia, Azospirillum, Dechlorospirillum, Defluvicoccus, Inquilinus, Magnetospirillum, Phaeospirillum, Rhodocista, Rhodospira, Rhodospirillum, Rhodovibrio, Roseospira, Skermanella, Thalassospira, Tistrella, Acetobacter, Acidicaldus, Acidiphilium, Acidisphaera, Acidocella, Acidomonas, Asaia, Belnapia, Craurococcus, Gluconacetobacter, Gluconobacter, Kozakia, Leahibacter, Muricoccus, Neoasaia, Oleomonas, Paracraurococcus, Rhodopila, Roseococcus, Rubritepida, Saccharibacter, Stella, Swaminathania, Teichococcus, Zavarzinia, Rickettsia, Orientia, Wolbachia, Aegyptianella, Anaplasma, Cowdria, Ehrlichia, Neorickettsia, Caedibacter, Holospora, Lyticum, Odyssella, Symbiotes, Tectibacter, Blastomonas, Citromicrobium, Erythrobacter, Erythromicrobium, Kaistobacter, Lutibacterium, Novosphingobium, Porphyrobacter, Sandaracinobacter, Sphingobium, Sphingomonas, Sphingopyxis, Zymomonas, Achromobacter, Alcaligenes, Bordetella, Pelistega, Sutterella, Taylorella, Burkholderia, Chitinimonas, Cupriavidus, Lautropia, Limnobacter, Pandoraea, Paucimonas, Polynucleobacter, Ralstonia, Thermothrix, Acidovorax, Aquabacterium, Brachymonas, Comamonas, Curvibacter, Delftia, Hydrogenophaga, Ideonella, Leptothrix, Limnohabitans, Pelomonas, Polaromonas, Rhodoferax, Roseateles, Sphaerotilus, Tepidimonas, Thiomonas, Variovorax, Collimonas, Duganella, Herbaspirillum, Herminiimonas, Janthinospirillum, Massilia, Naxibacter, Oxalobacter, Oxalicibacterium, Telluria, Borrelia, Brevinema, Cristispira, Spirochaeta, Spironema, Treponema, Brachyspira (Serpulina), Leptospira, Leptonema, Thermodesulfobacterium, Thermotoga, Verrucomicrobium, Prosthecobacter and Akkermansia. In one particular embodiment, the recombinant bacterial host cell is from the genus Escherichia and, in some additional embodiments, from the species Escherichia coli. In one particular embodiment, the recombinant bacterial host cell is from the genus Bacillus and, in some additional embodiments, from the species Bacillus subtilis. In one specific embodiment, the recombinant bacterial host cell is from the genus Lactobacillus.

In some embodiments, the recombinant microbial host cell comprises a genetic modification (e.g., a heterologous nucleic acid molecule) allowing the recombinant expression of the polypeptide having fumonisin amine oxidase activity. In such embodiment, a heterologous nucleic acid molecule encoding the polypeptide having fumonisin amine oxidase activity can be introduced in the microbial host cell to express the polypeptide having fumonisin amine oxidase activity. The expression of the polypeptide having fumonisin amine oxidase activity can be constitutive or induced (for example, by the supplementation of the culture medium with an inducing agent, for example, IPTG). The expression of the polypeptide having fumonisin amine oxidase activity can occur during the propagation phase and/or the fermentation phase or any other anaerobic growth of the recombinant microbial host cell.

The heterologous polypeptide of the present disclosure can be expressed inside the recombinant microbial host cell, e.g., intracellularly or intracellular form. The polypeptides of the present disclosure can be modified to remove, if any, signal peptide sequences present in the native amino acid sequence of the polypeptide to allow for an intracellular expression. In some embodiments, the polypeptides of the present disclosure can be modified to replace the signal sequence with a N-terminus modification (for example methionine at the N-terminus) to allow for an intracellular expression (as explained herein for N-terminus variants of the heterologous polypeptide). In some embodiments, the intracellularly expressed heterologous polypeptide includes a fumonisin amine oxidase derived from a Aspergillus niger set forth in SEQ ID NO: 5, 27, 28 or 29 a variant thereof or a fragment thereof.

The heterologous polypeptide of the present disclosure can be secreted and remain physically associated with the recombinant microbial host cell (e.g., a membrane-associated form). In an embodiment, at least one portion (usually at least one terminus) of the heterologous polypeptide is bound, covalently, non-covalently and/or electrostatically for example, to the cell wall (and in some embodiments to the cytoplasmic membrane) of the recombinant microbial host cell. For example, the heterologous polypeptide can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the heterologous polypeptide may not be directly bound to the cell membrane or cell wall (e.g., such as when binding occurs via a tethering moiety), the protein is nonetheless considered a “cell-associated” heterologous polypeptide according to the present disclosure.

In some embodiments, the polypeptide having fumonisin amine oxidase activity is a chimeric polypeptide of formula (I) or (II):

(NH₂)SS-FAO-L-TT(COOH) (I)

(NH₂)SS-TT-L-FAO(COOH) (II)

wherein:
FAO is the heterologous polypeptide having fumonisin amine oxidase activity;
L is present or absent and is an amino acid linker;
TT is present or absent and is an amino acid tethering moiety for associating the heterologous polypeptide to a cell wall or cell membrane of the recombinant microbial host cell;
SS is present or absent and is a signal sequence moiety;
(NH₂) indicates the amino terminus of the polypeptide;
(COOH) indicates the carboxyl terminus of the polypeptide; and
“-” is an amide linkage.

In embodiments in which the heterologous polypeptide is intended to be associated at the surface of the microbial host cell via a tethering moiety (e.g., in a tethered form) at the surface of the recombinant microbial host cell, it includes both the SS and the TT moieties. In other embodiments in which the heterologous polypeptide of the present disclosure is intended to be secreted. When the polypeptides are secreted, they are transported to outside of the cell, the chimeric heterologous polypeptides have a SS moiety but lack a TT moiety.

In some embodiments, the heterologous polypeptide can be expressed to be located at and associated to the cell wall of the recombinant yeast host cell. In some embodiments, the polypeptide is expressed to be located at and associated to the external surface of the cell wall of the host cell. Recombinant microbial host cells all have a cell wall (which includes a cytoplasmic membrane) defining the intracellular (e.g., internally-facing the nucleus) and extracellular (e.g., externally-facing) environments. The polypeptide can be located at (and in some embodiments, physically associated to) the external face of the recombinant microbial host's cell wall and, in further embodiments, to the external face of the recombinant microbial host's cytoplasmic membrane. In the context of the present disclosure, the expression “associated to the external face of the cell wall/cytoplasmic membrane of the recombinant yeast host cell” refers to the ability of the polypeptide to physically integrate (in a covalent or non-covalent fashion), at least in part, in the cell wall (and in some embodiments in the cytoplasmic membrane) of the recombinant microbial host cell.

In some embodiments, the heterologous polypeptides of the present disclosure can be expressed inside the recombinant yeast host cell, e.g., intracellularly. In such embodiments, the polypeptides having fumonisin amine oxidase activity of formula (I) or (II) lack the SS moiety, the L moiety and the TT moiety. The polypeptides of the present disclosure expressed intracellularly can be modified to remove, if any, signal peptide sequences present in the native amino acid sequence of the polypeptide to allow for an intracellular expression.

As indicated above, in some embodiments, the polypeptide includes one or a combination of signal peptide sequence(s) allowing the transport of the polypeptide outside the microbial host cell's wall. The signal sequence can simply be added to the polypeptide or replace the signal peptide sequence already present in the protein from which the fumonisin amine oxidase is derived. The signal sequence can be native or heterologous to the protein from which the fumonisin amine oxidase is derived. In some embodiments, one or more signal sequences can be used. In some embodiments, the one or more signal sequences are cleaved once the polypeptide is secreted. In some embodiments, the signal sequence is from the invertase protein (and can have, for example, the amino acid sequence of SEQ ID NO: 7, be a variant of the amino acid sequence of SEQ ID NO: 7 or be a fragment of the amino acid sequence of SEQ ID NO: 7); the AGA2 protein (and can have, for example, the amino acid sequence of SEQ ID NO: 8, be a variant of the amino acid sequence of SEQ ID NO: 8 or be a fragment of the amino acid sequence of SEQ ID NO: 8); or the α-mating factor protein (and can have, for example, the amino acid sequence of SEQ ID NO: 9, be a variant of the amino acid sequence of SEQ ID NO: 9 or be a fragment of the amino acid sequence of SEQ ID NO: 9).

As indicated above, in some embodiments, the polypeptides include an amino acid tethering moiety (TT) which will provide or increase attachment to the cell wall of the recombinant host cell. In such embodiment, the chimeric polypeptide will be considered “tethered”. TT may increase or provide cell association to some polypeptides because they exhibit insufficient intrinsic cell association or simply lack intrinsic cell association. In some embodiments, the amino acid tethering moiety of the chimeric polypeptide is neutral with respect to the biological activity of the fumonisin amine oxidase polypeptide, e.g., does not interfere with the biological activity. In some embodiments, the association of the amino acid tethering moiety with the fumonisin amine oxidase polypeptide can increase the biological activity of fumonisin amine oxidase activity polypeptide (when compared to the non-tethered, “free” form). Various tethering amino acid moieties are known to the art and can be used in the chimeric proteins of the present disclosure. The tethering moiety can be a transmembrane domain found on another protein and allow the polypeptide to have a transmembrane domain. TT may be endogenous or exogenous to the host cell. In some embodiments, TT is endogenous to the host cell.

In some embodiments, TT is derived from a cell surface protein, such as a glycosylphosphotidylinositol (GPI) associated anchor protein. GPI anchors are glycolipids attached to the terminus of a protein (and in some embodiments, to the carboxyl terminus of a protein) which allows the anchoring of the protein to the cytoplasmic membrane of the cell membrane. Tethering amino acid moieties capable of providing a GPI anchor include, but are not limited to those associated with/derived from a SED1 protein (having, for example, the amino acid sequence of SEQ ID NO: 10, a variant thereof or a fragment thereof), a SPI1 protein (having, for example, the amino acid sequence of SEQ ID NO: 11, a variant thereof or a fragment thereof), a CCW12 protein (having, for example, the amino acid sequence of SEQ ID NO: 12, a variant thereof or a fragment thereof), a CWP2 protein (having, for example, the amino acid sequence of SEQ ID NO: 13, a variant thereof or a fragment thereof), a TIR1 protein (having, for example, the amino acid sequence of SEQ ID NO: 14, a variant thereof or a fragment thereof), a PST1 protein (having, for example, the amino acid sequence of SEQ ID NO: 15, a variant thereof or a fragment thereof) or a combination of a AGA1 and a AGA2 protein (having, for example, the amino acid sequence of SEQ ID NO: 16, a variant thereof or a fragment thereof or having, for example, the amino acid sequence of SEQ ID NO: 17, a variant thereof or a fragment thereof).

In some embodiments, TT can comprise a transmembrane domain, a variant or a fragment thereof. For example, the tethering moiety can be derived from the FLO1 protein (having, for example, the amino acid sequence of SEQ ID NO: 18, a variant thereof or a fragment thereof).

Still in the context of the present disclosure, TT includes variants of the tethering moieties, such as, for example, variants of SEQ ID NOs: 10, 11, 12, 13, 14, 15, 16, 17, and 18 (also referred to herein as TT variants). A variant comprises at least one amino acid difference (substitution or addition) when compared to the amino acid sequence of the original tethering moiety and is capable locating a polypeptide to the membrane of the yeast cell. The TT variants exhibit cell wall anchoring activity. In an embodiment, the TT variant exhibits at least 50%, 60%, 70%, 80%, 90%, 95%, 96%, 97%, 98% or 99% of the cell wall anchoring activity of the amino acid of any one of SEQ ID NOs: 10, 11, 12, 13, 14, 15, 16, 17, and 18. The TT variants also have at least 70%, 80%, 85%, 90%, 95%, 96%, 97%, 98% or 99% identity to the amino acid sequence of any one of SEQ ID NOs: 10, 11, 12, 13, 14, 15, 16, 17, and 18.

The TT variants described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A TT variant can be also be a conservative variant or an allelic variant.

The present disclosure also provide fragments of TT and TT variants described herein. A fragment comprises at least one less amino acid residue when compared to the amino acid sequence of the TT polypeptide or variant and still possess the cell wall anchoring activity of the full-length TT portion. In an embodiment, the TT fragment exhibits at least 50%, 60%, 70%, 80%, 90%, 95%, 96%, 97%, 98% or 99% of the cell wall anchoring activity of the amino acid of any one of SEQ ID NOs: 10, 11, 12, 13, 14, 15, 16, 17 or 18. The TT fragments can also have at least 70%, 80%, 85%, 90%, 95%, 96%, 97%, 98% or 99% identity to the amino acid sequence of any one of SEQ ID NO: 10 to 18. The TT fragment can be, for example, a truncation of one or more amino acid residues at the amino-terminus, the carboxy-terminus or both termini of the polypeptide having fumonisin amine oxidase activity or variant. Alternatively or in combination, the fragment can be generated from removing one or more internal amino acid residues. In an embodiment, the TT fragment has at least 10, 20, 30, 40, 50, 60, 70, 80, 90, 100, 150, 200, 250, 300, 350, 400, 450, 500, 550, 600, 650 or more consecutive amino acids of the TT portion polypeptide or the variant.

In some embodiments, the TT is a fragment of a SPI1 protein. The fragment of the SPI1 protein comprises less than 129 amino acid consecutive residues of the amino acid sequence of SEQ ID NO: 11. For example, the TT fragment is from the SPI1 protein and can comprise at least 10, 20, 21, 30, 40, 50, 51, 60, 70, 80, 81, 90, 100, 110, 111 or 120 consecutive amino acid residues from the amino acid sequence of SEQ ID NO: 11.

In some embodiments, the TT is a fragment of a CCW12 protein. The fragment of the CCW12 protein comprises less than 112 amino acid consecutive residues of the amino acid sequence of SEQ ID NO: 12. For example, the TT fragment from the CCW12 protein can comprise at least 10, 20, 24, 30, 40, 49, 50, 60, 70, 74, 80, 90, 99, 100 or 110 consecutive amino acid residues from the amino acid sequence of SEQ ID NO: 12.

In embodiments in which the amino acid linker (L) is absent from the polypeptides of formula (I) and (II), the tethering amino acid moiety is directly associated with the heterologous protein. In the chimeras of formula (I), this means that the carboxyl terminus of the heterologous polypeptide moiety is directly associated (with an amide linkage) to the amino terminus of the tethering amino acid moiety. In the chimeras of formula (II), this means that the carboxyl terminus of the tethering amino acid moiety is directly associated (with an amide linkage) to the amino terminus of the heterologous protein.

In some embodiments, the presence of an amino acid linker (L) is desirable either to provide, for example, some flexibility between the heterologous protein moiety and the tethering amino acid moiety or to facilitate the construction of the heterologous nucleic acid molecule. As used in the present disclosure, the “amino acid linker” or “L” refer to a stretch of one or more amino acids separating the fumonisin amine oxidase polypeptide FAO and the amino acid tethering moiety TT (e.g., indirectly linking the fumonisin amine oxidase polypeptide to the amino acid tethering moiety TT). It is preferred that the amino acid linker be neutral, e.g., does not interfere with the biological activity of the heterologous protein nor with the biological activity of the amino acid tethering moiety. In some embodiments, the amino acid linker L can increase the biological activity of the fumonisin amine oxidase polypeptide and/or of the tethering moiety.

In instances in which the linker (L) is present in the chimeras of formula (I), its amino end is associated (with an amide linkage) to the carboxyl end of the heterologous protein moiety and its carboxyl end is associated (with an amide linkage) to the amino end of the amino acid tethering moiety. In instances in which the linker (L) is present in the chimeras of formula (II), its amino end is associated (with an amide linkage) to the carboxyl end of the amino acid tethering moiety and its carboxyl end is associated (with an amide linkage) to the amino end of the heterologous protein moiety. Various amino acid linkers exist and include, without limitations, (GS)ₙ; (GGS)ₙ; (GGGS)ₙ; (GGGGS)ₙ; (GGSG)ₙ; (GSAT)ₙ, wherein n=is an integer between 1 to 8 (or more). In an embodiment, the amino acid linker L is (GGGGS)ₙ (also referred to as G₄S) and, in still further embodiments, the amino acid linker L comprises more than one G₄S motifs. In some embodiments, L is chosen from: (G₄S)₃ (SEQ ID NO: 19), (G)₈ (SEQ ID NO: 20), (G₄S)₈ (SEQ ID NO: 21), GSAGSAAGSGEF (SEQ ID NO: 22), (EAAK)₃ (SEQ ID NO: 23), (AP)₁₀ (SEQ ID NO: 24) and A(EAAAK)₄ALEA(EAAAK)₄A (SEQ ID NO: 25). In some embodiments, the linker also includes one or more HA tag (SEQ ID NO: 26).

Nucleic acid molecules for expressing the heterologous polypeptides having fumonisin amine oxidase activity

In some embodiments, the nucleic acid molecules encoding the heterologous polypeptides, fragments or variants that can be introduced into the recombinant microbial host cells are codon-optimized with respect to the intended recipient recombinant host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0. An embodiment of a codon-optimized nucleic acid molecule for expression in Escherichia coli is the nucleic acid molecule having the nucleic acid sequence of SEQ ID NO: 6. An embodiment of a codon-optimized nucleic acid molecule for expression in Saccharomyces cerevisiae is the nucleic acid molecule having the nucleic acid sequence of SEQ ID NO: 37.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleotide acid molecule coding for the polypeptide in a manner that allows, under certain conditions, for expression of the peptide from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous polypeptide. In the context of the present disclosure, one or more than one promoter can be included in the nucleic acid molecule. When more than one promoter is included in the nucleic acid molecule, each of the promoters is operatively linked to the nucleic acid sequence coding for the polypeptide. The promoters can be located, in view of the nucleic acid molecule coding for the polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same, or species of the yeast host cell and the polypeptide is derived from different genera that the host cell. One or more promoters can be used to allow the expression of the polypeptides in the recombinant yeast host cell.

In some embodiments, the host is a facultative anaerobe, such as S. cerevisiae. For facultative anaerobes, cells tend to propagate or ferment depending on the availability of oxygen. In a fermentation process, yeast cells are generally allowed to propagate before fermentation is conducted. In some embodiments, the promoter preferentially initiates transcription during a propagation phase such that the polypeptides are expressed during the propagation phase. As used in the context of the present disclosure, the expression “propagation phase” refers to an expansion phase of a commercial process in which the yeasts are propagated under aerobic conditions to maximize the conversion of a substrate into biomass. In some instances, the propagated biomass can be used in a following fermenting step (e.g., under anaerobic conditions) to maximize the production of one or more desired metabolites.

In some embodiments, the promoter or the combination of promoters present in the heterologous nucleic acid is capable of allowing the expression of the recombinant heterologous polypeptide during the propagation phase of the recombinant microbial host cell. This will allow the accumulation of the polypeptide associated with the recombinant microbial host cell prior to any subsequent use, for example in liquefaction or fermentation. In some embodiments, the promoter allows the expression of the polypeptide during the propagation phase.

In other embodiments, the promoter or the combination of promoters present in the heterologous nucleic acid is capable of allowing the expression of the recombinant heterologous polypeptide during the anaerobic growth or culture (for example, in the fermentation phase) of the recombinant microbial host cell.

The promoters that can be included in the heterologous nucleic acid molecule can be constitutive or inducible promoters. Inducible promoters include, but are not limited to glucose-regulated promoters (e.g., the promoter of the hxt7 gene (referred to as hxt7p), a functional variant or a functional fragment thereof; the promoter of the ctt1 gene (referred to as ctt1p), a functional variant or a functional fragment thereof; the promoter of the glo1 gene (referred to as glo1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), molasses-regulated promoters (e.g., the promoter of the mol1 gene (referred to as mol1p), a functional variant or a functional fragment thereof), heat shock-regulated promoters (e.g., the promoter of the glo1 gene (referred to as glo1p), a functional variant or a functional fragment thereof; the promoter of the sti1 gene (referred to as sti1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), oxidative stress response promoters (e.g., the promoter of the cup1 gene (referred to as cup1p), a functional variant or a functional fragment thereof; the promoter of the ctt1 gene (referred to as ctt1p), a functional variant or a functional fragment thereof; the promoter of the trx2 gene (referred to as trx2p), a functional variant or a functional fragment thereof; the promoter of the gpd1 gene (referred to as gpd1p), a functional variant or a functional fragment thereof; the promoter of the hsp12 gene (referred to as hsp12p), a functional variant or a functional fragment thereof), osmotic stress response promoters (e.g., the promoter of the ctt1 gene (referred to as ctt1p), a functional variant or a functional fragment thereof; the promoter of the glo1 gene (referred to as glo1p), a functional variant or a functional fragment thereof; the promoter of the gpd1 gene (referred to as gpd1p), a functional variant or a functional fragment thereof; the promoter of the ygp1 gene (referred to as ygp1p), a functional variant or a functional fragment thereof), nitrogen-regulated promoters (e.g., the promoter of the ygp1 gene (referred to as ygp1p), a functional variant or a functional fragment thereof) and the promoter of the adh1 gene (referred to as adh1p), a functional variant or a functional fragment thereof.

Promoters that can be included in the heterologous nucleic acid molecule of the present disclosure include, without limitation, the promoter of the tdh1 gene (referred to as tdh1p, a functional variant or a functional fragment thereof), of the hor7 gene (referred to as hor7p, a functional variant or a functional fragment thereof), of the hsp150 gene (referred to as hsp150p, a functional variant or a functional fragment thereof), of the hxt7 gene (referred to as hxt7p, a functional variant or a functional fragment thereof), of the gpm1 gene (referred to as gpm1p, a functional variant or a functional fragment thereof), of the pgk1 gene (referred to as pgk1p, a functional variant or a functional fragment thereof), of the stl1 gene (referred to as stl1p, a functional variant or a functional fragment thereof) and/or of the tef2 gen (referred to as tef2p, a functional variant or a functional fragment thereof).

Promoters that can be included in the heterologous nucleic acid molecule of the present disclosure include, without limitation, phage-derived promoters, such as the T5 or the T7 promoter. These promoters are particularly useful for the expression of the polypeptide having fumonisin amine oxidase activity in a bacterial host cell, such as Escherichia coli.

In the context of the present disclosure, the expression “functional fragment of a promoter” refers to a shorter nucleic acid sequence than the native promoter which retain the ability to control the expression of the nucleic acid sequence encoding the heterologous polypeptides. Usually, functional fragments are either 5′ and/or 3′ truncation of one or more nucleic acid residue from the native promoter nucleic acid sequence.

In the context of the present disclosure, the expression “functional fragment of a promoter” refers to a nucleic acid sequence which differs in at least one position and still retain the ability to control the expression of the nucleic acid sequence encoding the heterologous polypeptide.

In some embodiments, the heterologous nucleic acid molecules include one or a combination of terminator sequence(s) to end the translation of the heterologous protein (or of the chimeric protein comprising same). The terminator can be native or heterologous to the nucleic acid sequence encoding the heterologous protein or its corresponding chimera. In some embodiments, one or more terminators can be used. In some embodiments, the terminator comprises the terminator derived from is from the dit1 gene (dit1t, a functional variant or a functional fragment thereof), from the idpl gene (idplt, a functional variant or a functional fragment thereof), from the gpm1 gene (gpm1t, a functional variant or a functional fragment thereof), from the pma1 gene (pam1t, a functional variant or a functional fragment thereof), from the tdh3 gene (tdh3t, a functional variant or a functional fragment thereof), from the hxt2 gene (a functional variant or a functional fragment thereof), from the adh3 gene (adh3t, a functional variant or a functional fragment thereof), and/or from the ira2 gene (ira2t, a functional variant or a functional fragment thereof). In an embodiment, the terminator comprises or is derived from the dit1 gene (dit1t, a functional variant or a functional fragment thereof). In another embodiment, the terminator comprises or is derived adh3t and/or idplt. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous protein or its corresponding chimera.

The heterologous nucleic acid molecules of the present disclosure can also include a portion encoding a signal sequence which is operatively linked to the portion encoding the heterologous polypeptide having fumonisin amine oxidase. The nucleic acid portion encoding the signal sequence is usually located 3′ to the promoter and 5′ to the portion encoding the heterologous polypeptide having fumonisin amine oxidase. The heterologous nucleic acid molecules, especially designed to be used in eukaryotic cells, can also include a 5′ untranslated region (UTR) between the one or more promoters and the heterologous polypeptide reading frame. In some embodiments, the 5′ UTR is associated with or derived from the one or more promoters used in the heterologous nucleic acid molecule.

Microbial Compositions

The present disclosure provides microbial compositions including the heterologous polypeptide having fumonisin amine oxidase activity described herein. The microbial compositions can also include the recombinant microbial host cell (living or dead) or at least one component of the recombinant microbial host cell. The “at least one component” can be an intracellular component and/or a component associated with the microbial host cell's wall or membrane. The “at least one component” can include a protein, a peptide or an amino acid, a carbohydrate and/or a lipid. The “at least one component” can include a microbial host cell organelle. The “at least one component” can be a microbial extract, such as, for example, a bacterial extract, a fungal extract or a yeast extract. The microbial composition can be an inactive product (e.g., none of the recombinant microbial host cell are alive), a semi-active product (e.g., some of the recombinant microbial host cells are alive) or an active product (e.g., most of the recombinant microbial host cells are alive). Inactivated yeast products include, but are not limited to a yeast extract and an active/semi-active yeast products include, but are not limited to, a cream yeast. Inactivated bacterial products, include but are not limited to a bacterial extract and an active/semi-active bacterial products include, but are not limited to, bacterial concentrates. Inactivated fungal products, include but are not limited to a fungal extract and an active/semi-active fungal products include, but are not limited to, fungal concentrates. In some embodiments, the yeast product is a yeast extract produced from recombinant yeast host cells expressing the polypeptides. In some additional embodiment, the bacterial product is a bacterial extract produced from the recombinant microbial host cells expressing the polypeptides. In some additional embodiment, the fungal product is a fungal extract produced from the recombinant microbial host cells expressing the polypeptides. The recombinant microbial cell of the microbial composition can be frozen or dehydrated (e.g., lyophilized).

The microbial composition can also be an isolated, synthetic or recombinant heterologous polypeptide having fumonisin amine oxidase activity. In such embodiment, the isolated, synthetic or recombinant heterologous polypeptide having fumonisin amine oxidase activity has been produced from the recombinant microbial host cell and substantially isolated or purified therefrom. As used in the context of the present disclosure, the expressions “purified form” or “isolated form” refers to the fact that the polypeptides have been physically dissociated from at least one components required for their production (such as, for example, a host cell or a host cell fragment). A purified form of the heterologous polypeptide of the present disclosure can be a cellular extract of a host cell expressing the polypeptide being enriched for the polypeptide of interest (either through positive or negative selection). The expressions “substantially purified form” or “substantially isolated” refer to the fact that the polypeptides have been physically dissociated from the majority of components required for their production (including, but not limited to, components of the recombinant yeast host cells). In an embodiment, an heterologous polypeptide in a substantially purified form is at least 90%, 95%, 96%, 97%, 98% or 99% pure.

As used in the context of the present disclosure, the expression “recombinant form” refers to the fact that the polypeptides have been produced by recombinant DNA technology using genetic engineering to express the polypeptides in the recombinant yeast host cell.

The microbial composition can be provided in a liquid, semi-liquid or dry form. The microbial composition can be a bacterial composition. The microbial composition can be a yeast composition. The microbial composition can be a fungal composition.

The present disclosure also includes a process for making the isolated, synthetic or polypeptide having heterologous fumonisin amine oxidase activity. First, the recombinant microbial host cells described herein must be propagated to increase the biomass and favor the expression of the heterologous polypeptide having fumonisin amine oxidase activity. The propagation step is usually conducted in a culture medium allowing the propagation of the recombinant microbial host cell under conditions (agitation, temperature, etc.) so as to favor the expression of the heterologous polypeptide having fumonisin oxidase activity. Once the recombinant microbial host cells have been propagated, they can optionally be submitted to an anaerobic growth phase (such as a fermentation phase). The propagated and optionally fermented microbial host cells then are submitted to a dissociation step or a lysis step to obtain a dissociated fraction enriched in the heterologous polypeptide or a lysed fraction. When the heterologous polypeptide is expressed in a secreted form, the dissociation step can include, for example, a filtration or a centrifugation step to obtain the dissociated fraction. When the heterologous polypeptide is expressed intracellularly or associated with the membrane, the recombinant microbial host cells can be lysed to obtain the lysed fraction and facilitate downstream processing. The lysis step can be achieved, for example, by autolysis, a heat treatment, a pH treatment, a salt treatment, an homogenization step, etc. The process can include, in some embodiments, drying the dissociated or lysed fraction obtained prior to the purification step. The process further includes a step of substantially purifying the heterologous polypeptide having fumonisin oxidase activity from the dissociated, lysed or dried fraction. The process can include one or more washing steps and/or a further dried step after the purification step. The process can include determining the purity or the activity of the isolated, synthetic or recombinant heterologous polypeptide having fumonisin amine oxidase activity.

The process can also be used to make a yeast product. When the yeast product is an inactivated yeast product, the process for making the yeast product broadly comprises two steps: a first step of providing propagated recombinant yeast host cells and a second step of lysing the propagated yeast host cells for making the yeast product. The process for making the yeast product can include an optional separating step and an optional drying step. In some embodiments, the propagated recombinant yeast host cells are propagated on molasses. Alternatively, the propagated recombinant yeast host cells are propagated on a medium comprising a yeast extract.

The process can also be used to make a bacterial product. When the bacterial product is an inactivated bacterial product, the process for making the bacterial product broadly comprises two steps: a first step of providing propagated recombinant bacterial host cells and a second step of lysing the propagated bacterial host cells for making the yeast product. The process for making the bacterial product can include an optional separating step and an optional drying step. In some embodiments, the propagated recombinant bacterial host cells are on a medium comprising a yeast extract.

The process can also be used to make a fungal product. When the fungal product is an inactivated fungal product, the process for making the fungal product broadly comprises two steps: a first step of providing propagated recombinant fungal host cells and a second step of lysing the propagated fungal host cells for making the fungal product. The process for making the fungal product can include an optional separating step and an optional drying step. In some embodiments, the propagated recombinant fungal host cells are propagated on molasses. Alternatively, the propagated recombinant fungal host cells are propagated on a medium comprising a fungal extract.

In some embodiments, the recombinant yeast host cells can be lysed using autolysis (which can optionally be performed in the presence of additional exogenous enzymes). For example, the propagated recombinant yeast host cells may be subject to a combined heat and pH treatment for a specific amount of time (e.g., 24 h) in order to cause the autolysis of the propagated recombinant yeast host cells to provide the lysed recombinant yeast host cells. For example, the propagated recombinant yeast host cells can be submitted to a temperature of between about 40° C. to about 70° C. or between about 50° C. to about 60° C. The propagated recombinant yeast host cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant yeast host cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant yeast host cells can be submitted to a pH between about 4.0 and 8.5 or between about 5.0 and 7.5. The propagated recombinant yeast host cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant yeast host cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5.

In some embodiments, the recombinant yeast host cells can be homogenized (for example using a bead-milling technique, a bead-beating or a high pressure homogenization technique) and as such the process for making the yeast product comprises an homogenizing step.

In some embodiments, the recombinant bacterial host cells can be homogenized (for example using a bead-milling technique, a bead-beating or a high pressure homogenization technique) and as such the process for making the bacterial product comprises an homogenizing step.

In some embodiments, the recombinant fungal host cells can be homogenized (for example using a bead-milling technique, a bead-beating or a high pressure homogenization technique) and as such the process for making the fungal product comprises an homogenizing step.

The process for making the yeast product can also include a drying step. The drying step can include, for example, with spray-drying and/or fluid-bed drying. When the yeast product is an autolysate, the process may include directly drying the lysed recombinant yeast host cells after the lysis step without performing an additional separation of the lysed mixture.

The process for making the bacterial product can also include a drying step. The drying step can include, for example, with spray-drying and/or fluid-bed drying. When the bacterial product is an autolysate, the process may include directly drying the lysed recombinant bacterial host cells after the lysis step without performing an additional separation of the lysed mixture.

The process for making the fungal product can also include a drying step. The drying step can include, for example, with spray-drying and/or fluid-bed drying. When the fungal product is an autolysate, the process may include directly drying the lysed recombinant fungal host cells after the lysis step without performing an additional separation of the lysed mixture.

To provide additional yeast products, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction”) of the lysed recombinant yeast host cells. This separating step can be done, for example, by using centrifugation and/or filtration. The process of the present disclosure can include one or more washing step(s) to provide the cell walls or the yeast extract. The yeast extract can be made by drying the soluble fraction obtained.

In an embodiment of the process, the soluble fraction can be further separated prior to drying. For example, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried to provide a retentate as the yeast product.

When the yeast composition is an active/semi-active product, it can be submitting to a concentrating step, e.g. a step of removing part of the propagation/fermentation medium from the propagated recombinant yeast host cells. The concentrating step can include resuspending the concentrated and propagated/fermented recombinant yeast host cells in the propagation medium (e.g., unwashed preparation) or a fresh medium or water (e.g., washed preparation).

When the bacterial composition is an active/semi-active product, it can be submitting to a concentrating step, e.g. a step of removing part of the propagation/fermentation medium from the propagated recombinant bacterial host cells. The concentrating step can include resuspending the concentrated and propagated recombinant bacterial host cells in the propagation/fermentation medium (e.g., unwashed preparation) or a fresh medium or water (e.g., washed preparation).

When the fungal composition is an active/semi-active product, it can be submitting to a concentrating step, e.g. a step of removing part of the propagation/fermentation medium from the propagated/fermented recombinant fungal host cells. The concentrating step can include resuspending the concentrated and propagated recombinant fungal host cells in the propagation/fermentation medium (e.g., unwashed preparation) or a fresh medium or water (e.g., washed preparation).

In an aspect, the heterologous polypeptides having fumonisin amine oxidase activity may be provided in a composition that additionally includes a culture medium (used or intended to be used with the microbial host cell).

Methods of Using the Heterologous Polypeptide Having Fumonisin Amine Oxidase Activity

The heterologous polypeptide having fumonisin amine oxidase activity of the present disclosure can be used to detoxify a fumonisin, especially a fumonisin bearing one or more tricarballylic ester substituent. Fumonisins are found in various feed and food components. Fumonisins can be found, for example, in silage (maize, grass, sorghum, sweet potato vines for example), hay, straw, grains (maize, oat, wheat, rye, barley, rice for example), grain by-products (distillers grains for examples), legumes (peanut and soybean for example), cottonseed meal, vegetables (cabbage, carrots, corn for example), fruits, milk, milk by-products (whey for example) as well as in commercial animal feed products. The heterologous fumonisin amine oxidase of the present disclosure can be used to detoxify contaminated feed and food components. As used in the context of the present application, the term “detoxify a fumonisin mycotoxin” refers to the ability of the heterologous polypeptide having fumonisin oxidase activity to cause the deamination of the fumonisin mycotoxin into an oxidized fumonisin mycotoxin. As indicated herein, in its oxidized form, the fumonisin mycotoxin is less toxic than in its amine form. In some embodiments, the methods can be used to convert at least 5, 10, 15, 20, 25, 30, 35, 40, 45, 50, 55, 60, 65, 70, 75, 80, 85, 90, 95% or more of the fumonisin mycotoxin into its oxidized (less toxic) form.

The method includes a step of contacting the heterologous polypeptide having fumonisin amine oxidase activity (either in an isolated, synthetic or recombinant form or in a microbial composition (in the presence of recombinant microbial host cell or a component thereof)) with the fumonisin mycotoxin under conditions so as to allow the deamination of the mycotoxin. The method can thus include a step of contacting a food or feed components with the polypeptide having fumonisin amine oxidase activity within silage (maize, grass, sorghum, sweet potato vines for example), hay, straw, grains (maize, oat, wheat, rye, barley, rice for example), grain by-products (distillers grains for examples), legumes (peanut and soybean for example), cottonseed meal, fruits, vegetables (cabbage, carrots, corn for example), milk, milk by-products (whey for example) as well as in commercial animal feed and human food products. The contacting step can be conducted under a certain temperature or temperature range. For example, the contacting step can be conducted at a temperature higher than 4° C. and lower than 95° C. In an embodiment, the contacting step is conducted at a temperature of at least 5, 10, 15, 20, 25, 30, 35, 40, 45, 50, 55, 60, 65, 70, 75, 80, 85 or 90° C. In another embodiment, the contacting step is conducted at a temperature of no more than 90, 85, 80, 75, 70, 65, 60, 55, 50, 45, 40, 35, 30, 25, 20, 15, 10 or 5° C. In yet another embodiment, the contacting step is conducted at a temperature between 5, 10, 15, 20, 25, 30, 35, 40, 45, 50, 55, 60, 65, 70, 75, 80, 85 or 90° C. and 90, 85, 80, 75, 70, 65, 60, 55, 50, 45, 40, 35, 30, 25, 20, 15, 10 or 5° C. In a further embodiment, the contacting step is conducted at a temperature between 20 and 40° C., for example, at a temperature of 37° C. The contacting step can be conducted at a certain pH or pH range. For example, the contacting step can be conducted at a pH of at least 3, 4, 5, 6, 7 or 8. In another example, the contacting step can be conducted at a pH of no more than 8, 7, 6, 5, 4 or 3. In still another example, the contacting step can be conducted at a pH between 3 and 8, for example, between a pH of at least 3, 4, 5, 6, 7 or 8 and a pH of no more than 8, 7, 6, 5, 4 or 3. In a specific example, the contacting step can be conducted at a pH between 5 and 7, for example, at a pH of 6. In another embodiment, the contacting step is conducted at a temperature of 37° C. and a pH of 6. The contacting can be done with directly with solid components. Alternatively, the contacting can be done when the food or feed components is in contact with a liquid, such as, for example, water.

In an embodiment, the method includes a step of determining if the feed or food components are contaminated with the fumonisin mycotoxin either prior to and/or after the contact with the heterologous polypeptide having fumonisin amine oxidase activity.

The method of the present disclosure can be applied to the detoxification of components to be included in feed or the feed itself. In an embodiment, the feed components are grains that have been submitted to a fermentation step (to convert the grains into a fermented product, like ethanol for example) are referred to a distillers grains. Distillers grains can be obtained during the fermentation of grains (such as corn for example) during the process for making distilled spirits or of biofuels. As it is known in the art, distillers grains have a high nutritional value and can be used as a feed product (alone or combined with other feed product or additives). The method described herein can be applied to distillers grain (either in a wet or dried form) to detoxify the fumonisin mycotoxin that may be present.

In an embodiment, distillers grain can be obtained via a process that comprises combining a substrate to be hydrolyzed (optionally included in a liquefaction medium) with a fermenting yeast cells (which could be the recombinant yeast host cells expressing the heterologous polypeptide having fumonisin amine oxidase activity) to perform a fermentation of the substrate. At this stage, further purified enzymes, such as, for example, alpha-amylases or glucoamylases can also be included in the liquefaction medium or the fermentation medium. The substrate can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and proline-rich proteins). The substrate comprises starch (in a gelatinized or raw form). In some embodiments, the substrate is derived from corn.

Once the fermentation has been completed, the fermented substrate is treated to purify or isolate the fermented product (for example ethanol) from the fermented substrate. When the fermenting agent is the recombinant yeast host cell having expressed and produced the polypeptide having fumonisin amine oxidase activity, the fermented substrate may have been detoxified during the liquefaction or fermentation and/or can be submitted to a detoxification step directly without the need of adding another source of the heterologous polypeptide having the fumonisin amine oxidase activity. In some embodiments, even when the fermenting agent is a recombinant yeast host cell having expressed and produced the heterologous polypeptide having fumonisin amine oxidase activity, it is necessary to add a further source of the heterologous polypeptide having fumonisin amine oxidase activity, either by adding the isolated, synthetic or recombinant heterologous polypeptide described herein, the microbial composition, the recombinant microbial host cell described herein or the microbial composition described herein to the fermented substrate to allow the detoxification of the fermented substrate.

In another embodiment, distillers grain can be obtained via a process for making an alcoholic beverage, such as beer or wine or a distilled spirit such as, for example, brandy as well as brandy-based wine, whisky, rum, vodka, gin, tequila, mexcal, sake, or arrack. In such process, a fermenting yeast (which can be the recombinant yeast host cell expressing the heterologous polypeptide having fumonisin amine oxidase) contacts the substrate and conducted a fermentation of the substrate. The liquid portion of the fermented substrate is submitted to a distillation step whereas the solid portion of the fermented substrate can serve as distillers grains. When the fermenting agent is a recombinant yeast host cell having expressed and produced the polypeptide having fumonisin amine oxidase activity, the solid portion of the fermented substrate may have been detoxified during the fermentation and/or can be submitted to a detoxification step directly without the need of adding another source of the heterologous polypeptide having the fumonisin amine oxidase activity. In some embodiments, even when the fermenting agent is a recombinant yeast host cell having expressed and produced the heterologous polypeptide having fumonisin amine oxidase activity, it may be necessary to add a further source of the heterologous polypeptide having fumonisin amine oxidase activity, either by adding the isolated, synthetic or recombinant heterologous polypeptide described herein, the recombinant microbial host cell described herein or the microbial composition described herein to the solid portion of the fermented substrate to allow the detoxification of the fermented substrate.

The detoxified fermented substrate or the detoxified solid portion of the fermented substrate can be further processed, as it is known in the art, to provide a feed product. For example, the method for making the feed can include adding an additive, such as, for example, yeast cell wall, a binder or a further mycotoxin-degrading enzyme to the detoxified substrate. The yeast cell wall additive can be provided from the recombinant yeast host cell or another yeast host cell.

In an embodiment, the product derived from the grains can be a food product. The food product includes grains or products derived from grains (such as flour for example), fruits, vegetables, or an alcoholic beverage. The food components can be detoxified prior to or after they have been processed into the food product. The detoxified grains can be crushed, grinded, sieved or filtered prior to or after the detoxification step. The food product can be further baked or fried.

The present disclosure also provides a feed or a food product comprising the isolated, synthetic or recombinant heterologous polypeptide having the fumonisin amine oxidase activity. In some embodiments, the feed and the food product also include a recombinant microbial host cell or at least one component derived therefrom. In some additional embodiments, the feed or the food product is obtained by the methods and processes described herein. The feed can be derived from distillers grain. The food product can be derived from grains and can be, for example, a flour. The flour can be a corn flour, a wheat flour, a barley flour, a buckwheat flour, a chickpea flour, etc.. The feed product of the present disclosure can also include an additive (e.g., yeast cell wall, a binder or a further mycotoxin-degrading enzyme).

The present invention will be more readily understood by referring to the following examples which are given to illustrate the invention rather than to limit its scope.

### EXAMPLE I

Enrichment of Fumonisin Deamination Activity from ASPERGILLUS

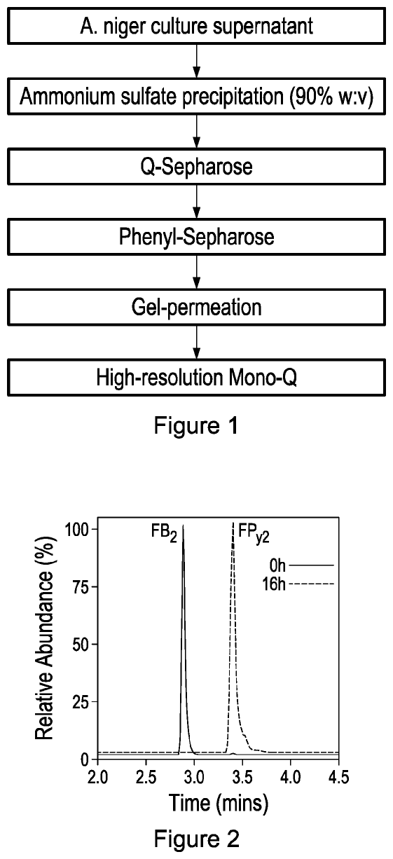

A protocol to enrich for fumonisin deamination activity from culture supernatants of Aspergillus was developed. The protocol consisted of a 90% (w:v) ammonium sulfate precipitation of the fungal culture supernatant, followed by Q-Sepharose™, Phenyl Sepharose™, gel permeation, and high resolution mono-Q™ chromatography steps performed on a Bio-Rad Fast Performance Liquid Chromatography (FPLC) system, as shown in FIG. 1.

After each step, protein fractions were assayed for deamination activity by monitoring their ability to convert intact FB₂ into FPy₂ via reverse-phase liquid chromatography/mass spectrometry (LC-MS), as shown in FIG. 2. In particular, all MS data were collected with a Q-Exactive™ Quadrupole Orbitrap™ mass spectrometer (Thermo Scientific, MA, USA) coupled to an Agilent 1290 ultra-high-performance liquid chromatography (UHPLC) system. Fumonisins were resolved on a Zorbax™ Eclipse Plus Rapid Resolution High Definition (RRHD) C18 column (2.1×50 mm, 1.8 μm; Agilent Technologies, CA, USA), maintained at 35° C. The mobile phase was comprised of water with 0.1% formic acid (A), and acetonitrile with 0.1% formic acid (B) (Optima grade, Fisher Scientific, NJ, USA). The gradient consisted of 0% B for 0.5 min before increasing to 100% over 3 min, held at 100% for 2.5 min and reduced to 0% over 0.5 min. The fumonisins were detected in negative ionization mode using the following electrospray conditions: capillary voltage, 4.0 kV; capillary temperature, 400° C.; sheath gas, 17.00 units; auxiliary gas, 8.00 units; probe heater temperature, 450° C.; S Lens RF level, 45.00. The data-dependent acquisition method involved a full MS scan at 17,500 resolution over a scan range of 140-760 m/z; automatic gain control (AGC) target and maximum injection time (max IT) was 5×10⁶ and 64 ms, respectively. The five highest intensity ions from the full scan (excluding isotopes) were sequentially selected using a 1.2 m/z isolation window and analyzed at a resolution of 17,500; AGC target, 1×10⁵; max IT, 64 ms; normalized collision energy (NCE) 30; threshold intensity 9.1×10⁴; and dynamic exclusion of 1.5 s. Fumonisins were detected by accurate mass (±5 ppm) and retention time (±0.1 min) and verified by MS/MS.

All samples assayed during protein purification were incubated at 37° C. with 1 μM FB₂ (Sigma) unless stated otherwise. Reactions were terminated via addition of a 10-fold volume excess of 50% (v:v) methanol in water prior to reverse-phase LC/MS analysis represented in FIG. 2.

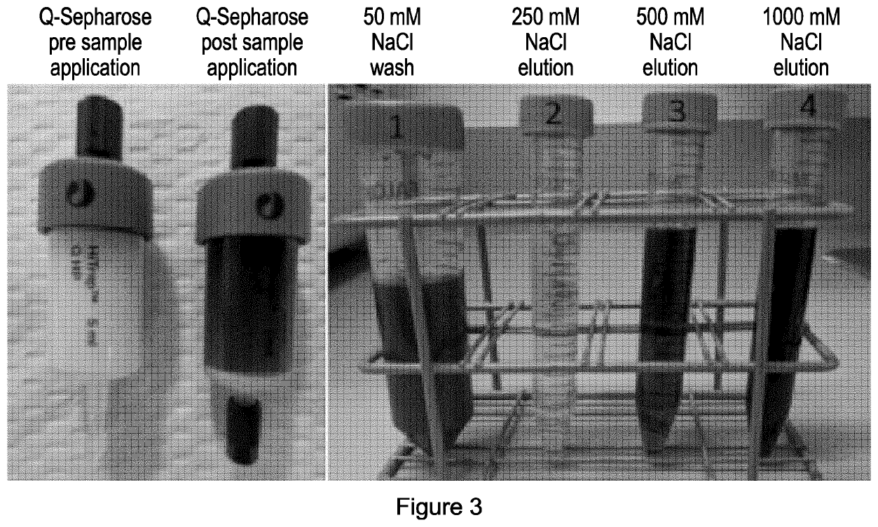

Following the 90% (w:v) ammonium sulfate precipitation, the pellet was re-suspended in 1/200^{th} the original total volume of the culture supernatant in buffer containing 50 mM 2-(N-morpholino)ethanesulfonic acid (MES) (pH 6), 50 mM NaCl (Buffer A). The re-suspended pellet was then dialyzed exhaustively against the same buffer to remove excess ammonium sulfate prior to Q-Sepharose™ chromatography. The dialyzed sample was then applied to a Q-Sepharose™ HP column (GE Healthcare) equilibrated in Buffer A. Fumonisin deamination activity bound to the column and was batch eluted in Buffer A containing 50 (fraction 1), 250 (fraction 2), 500 (fraction 3) or 1000 (fraction 4) mM NaCl. The 250 mM NaCl elution step isolated deamination activity from the majority of the contaminating black pigment and nucleic acids that eluted at higher NaCl concentrations as shown in FIG. 3.

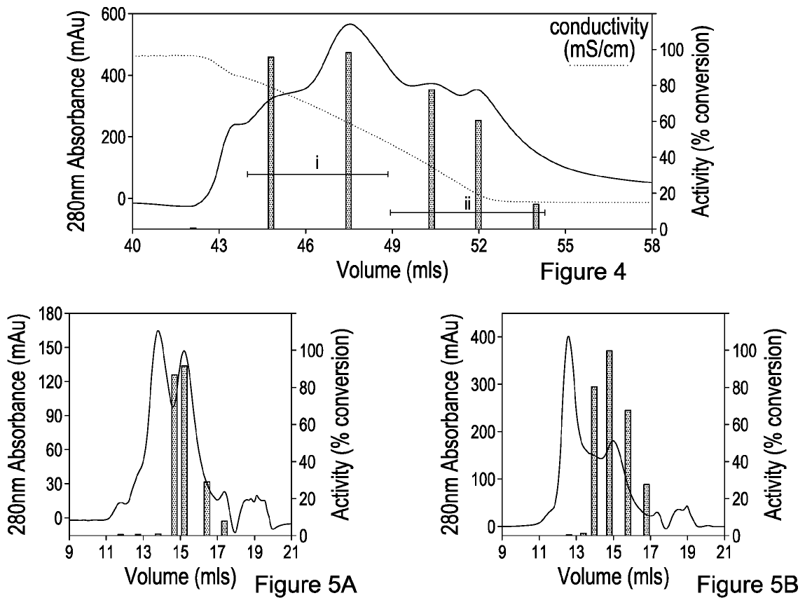

The eluted sample was then brought to 1 M ammonium sulfate and separated via hydrophobic interaction chromatography. A Phenyl HP column (GE Healthcare) was equilibrated in Buffer A containing 1 M ammonium sulfate. All deamination activity bound to the column and eluted broadly via decreasing [(NH₄)₂SO₄] gradient (1 M to 0 M) in Buffer A applied over 10 column volumes as shown in FIG. 4.

Due to the broad elution profile, the active fractions were split and pooled into two discrete samples as shown in FIGS. 5A and 5B, that spanned the first and second halves of the run and each were purified separately. Both were applied to an SEC650 gel permeation column (Bio-Rad) equilibrated in Buffer A.

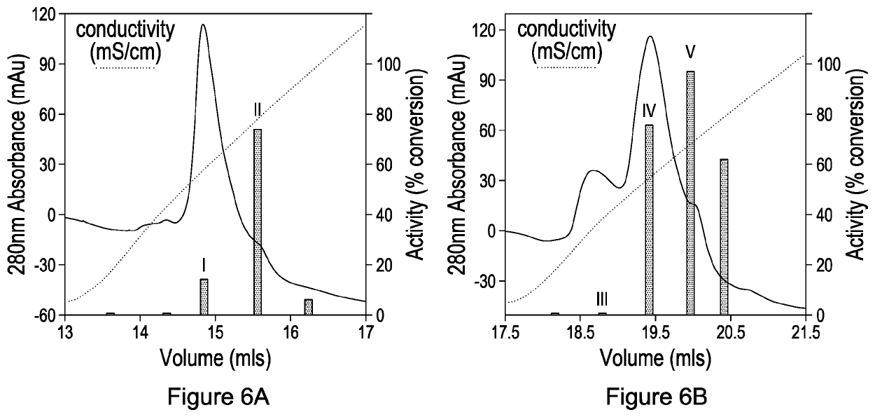

Deamination activity eluted discretely at ca. 0.6 column volumes during both runs. Finally, active fractions from both gel permeation runs were separately pooled and individually applied to a high-resolution mono-Q™ anion exchange column (Bio-Rad) equilibrated in Buffer A as shown in FIGS. 6A and 6B. All deamination activity bound to the column and eluted discretely in a shoulder of the main peak of each chromatogram when using an increasing NaCl gradient (0 M to 1 M) in Buffer A applied over 10 column volumes.

### EXAMPLE II

Characterization of Deamination Activity Isolated from ASPERGILLUS

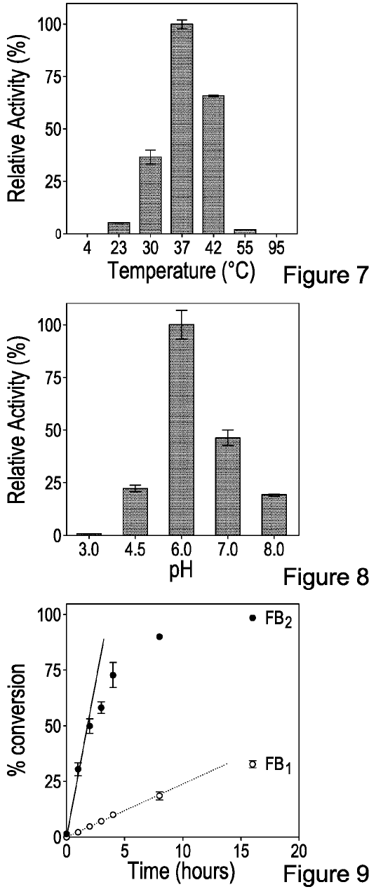

The temperature dependence of fumonisin deamination activity was tested by pre-incubating samples post ammonium sulfate precipitation for 20 minutes at temperatures of 4, 23, 30, 37, 42, 55, and 95° C. prior to addition of 1 μM FB₂. Samples were then incubated overnight at the same temperature prior to reverse phase LC-MS analysis. Fumonisin deamination activity occurred optimally at 37° C. and decreased uniformly as temperature was either raised or lowered, as shown in FIG. 7. Heating the sample to 95° C. completely abolished activity, while activity was also negligible at 4° C.

The pH-dependence of fumonisin deamination activity was also tested by adding concentrated buffer stock to each sample to a final concentration of 100 mM (ie: sodium citrate (pH 3), sodium citrate (pH 4.5), MES (pH 6), HEPES (pH 7), and Tris-HCl (pH 8.0). Each sample was then incubated overnight at 37° C. upon addition of 1 μM FB₁. Fumonisin deamination activity occurred optimally at a pH of 6, while activity was minimal at a pH of 3 and dropped to roughly 25% of maximum at pH 8.0, as shown in FIG. 8.

Finally, the fumonisin chemotype preference was tested by co-incubating both intact FB₁ and FB₂ at 37° C. with an active sample following high-resolution ion exchange. An order of magnitude preference for FB₂ compared to FB₁ was observed, as seen in FIG. 9.

The chemotype preference, pH- and temperature-dependence of the fumonisin deamination activity strongly indicated an enzyme was responsible for the detoxification.

### EXAMPLE III

Reverse Phase Lc-Ms/Ms to Identify Potential Fumonisin DEAMINATION ENZYMES

The identity of proteins present in fractions with fumonisin deaminating activity following high-resolution mono-Q™ anion exchange enrichment was determined via sequencing of tryptically-digested peptides by nanoLC-MS/MS. The proteins were enzymatically digested using the ThermoFisher SMART™ digest kit according to manufacturer's instructions. The peptide digests were analyzed using an Easy-nLC™ 1000 nano system with a 75 μm×15 cm Acclaim C18 PepMap™ column (Thermo Scientific) coupled to a Q-Exactive Orbitrap™ mass spectrometer (Thermo Fisher Scientific). The flow rate was 300 nL·min⁻¹ and 10 μL of the protein digest was injected. The C18 column was equilibrated with 98% mobile phase A (water+0.1% formic acid) and 2% mobile phase B (acetonitrile+0.1% formic acid) and eluted with a linear gradient from 2-30% B over 18 minutes followed by 30-98% B over 2 minutes and maintained for 10 minutes.

The nanospray voltage was set at 2.1 kV, capillary temperature 275° C., and S-lens RF level 55. The Q-Exactive was operated in top 5 data-dependent acquisition mode with a full scan mass range of 400 to 2000 m/z at 70,000 resolution, automatic gain control (AGC) of 1×10⁶ and maximum injection time (IT) of 250 ms. The MS/MS scans were acquired at 17,500 resolution, AGC of 2×10⁵, maximum IT of 50 ms, intensity threshold of 8×10⁴, normalized collision energy of 27 and isolation window of 1.2 m/z. Unassigned, singly and >4 charged peptides were not selected for MS/MS and a 20 s dynamic exclusion was used. The Thermo .raw files were converted to .mgf using Proteowizard v2 and the MS/MS scans were searched against the target/reverse UniProt Aspergillus niger (CBS 513.88) proteome using X! Tandem search algorithm operated from the SearchGUl v.2.35 interface and processed in PeptideShaker v1.3.6 (Vaudel et al., 2011; Vaudel et al., 2015). A 3 ppm precursor ion mass error and a 0.02 Da product ion error were used along with oxidation of methionine as a variable modification. A 1% FDR rate was used at the protein, peptide and peptide spectrum match level.

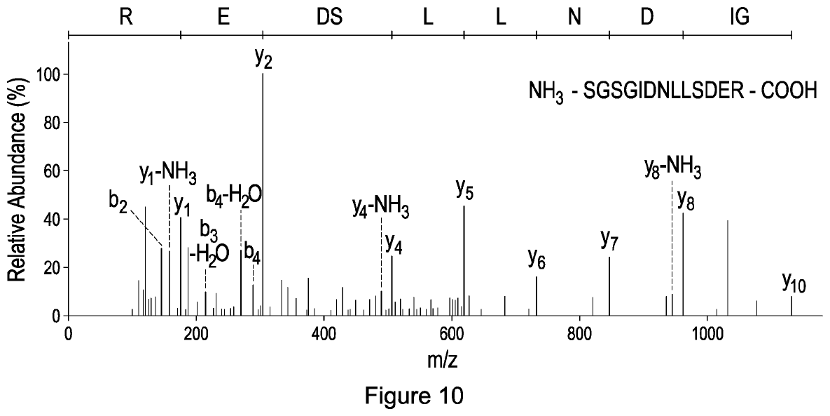

Five protein fractions were selected for proteomics analysis (see labelled peaks I-V, FIG. 6). In the sample with the highest deamination activity (peak V, FIG. 6B), a total of 60 proteins were identified with 100% confidence. Of these, any proteins that were co-observed between peaks III and V were eliminated as candidate enzymes due to the lack of deamination activity in fraction III. In addition, any proteins with a clearly defined known biological function, including but not limited to peptidases, hydrolases, esterases, and the like, were also eliminated as candidate deamination enzymes. Any proteins that were in greater abundance (as determined using spectrum counting) in Peak V compared to Peak IV were retained as candidate deamination enzymes, due to the higher fumonisin deamination activity of Peak V. This procedure produced five candidate enzymes, of which an amine oxidase (UniProtKB accession #A2R252, gene An13g03560) was the most likely candidate capable of oxidative deamination of fumonisins. Eleven unique peptides of the amine oxidase were observed, corresponding to ca. 24% coverage of the full-length enzyme. A representative spectrum, corresponding to the peptide (K)SGSGIDNLLSDER (SEQ ID NO: 1) (residues 194-206) of the amine oxidase is shown in FIG. 10. This same amine oxidase was also observed via proteomics in peaks I and II shown in FIG. 6A, and was in greater abundance in peak II compared to peak I.

### EXAMPLE IV

Recombinant Aspergillus Niger Amine Oxidase Deaminates FUMONISINS

Only approximately 25% of the amine oxidase open reading frame was observed via reverse phase LC-MS/MS peptide sequencing. The newly identified amine oxidase was therefore amplified from genomic DNA by polymerase chain reaction (PCR) using oligonucleotide primers designed to anneal upstream and downstream of the open reading frame. The sequence of the forward primer was 5′-CACTTCCTCAGCCTAATTTGC-3′ (SEQ ID NO: 2), and the sequence of the reverse primer was 5′-CTGGTGTAGATCTAACGAATA-C3′ (SEQ ID NO: 3). Fungal genomic DNA was isolated using the Dneasy™ UltraClean™ Microbial Kit (Qiagen) according to manufacturer's instructions and was used as template in a PCR reaction that successfully amplified a ca. 1733 base pair PCR product. This PCR product was sequenced and revealed the full open reading frame of the amine oxidase (referred to as the AnFAO_15309 clone).

The nucleotide sequence of the gene encoding the amine oxidase is:

(SEQ ID NO: 4)
ATGTCTGTATCCAACGATCCTACTACAAAGCTCTACGATGCGGTGATCG
TGGGAGCCGGACTTAGCGGCCTTCAAGCCGCGCATTCCATCCAGGCAGC
AGGATTCAGCGTGTGTATCCTGGAAGCTACGGACCGAATGGGTGGGAAG
ACGTTGACCGTAAAATCCAGCGAGAAAGGATACAACGATCTAGGAGCGG
CTTGGGTGAATGATACGAACCAGACGGAGATTTTCAAACTTCATCAGCG
GTATGGACTGGATGGGGTGGTTCAGTATACTTGTGGAGATGATATCCTC
GAGTCAGGCGAGGGGGTGATCCGCAAGATACCGTATGGATTGCCATTGA
CTGGGCTTCCGAAGAAATTGTTGGATATTCTCCGAATTGAGTCCTCACG
GTTAGACTTGGACGATCCTACGAGTTTTCCAGGGGCCACAGAAGTGGAT
AATCTGACGTTCAGGGACTTTTGCGTCGAGAAGACTGGATCGGAGGATG
TTATTCACATTACAGATGCTATTTCAACGGCGCTGCTTGGATTGAATAG
TAACGAACTCAGTGCTTTGTATATGCTCTACTACTTCAAAAGTGGGAGT
GGGATCGACAATCTGCTGTCAGATGAGAGAGACGGAGCACAGTACCTGC
GGACAAGACAAGGTACCCAAACCATCGCCCGGAAGATGGCAGATGAGCT
CACCCAATCGGACATTTTTCTGGGCATGCCCGTCACTTCAATCAATCAG
ACTGACGCTGATGCTCACTGCGTGGTCCAGACACTTGATGGAAGTTCTT
TTCGCTGTCGACGCGTTATCGTGTCTATCCCTACCACCTTATACCGGAG
TGTCTCCTTCCACCCCCCACTTCCACATGCAAAACAGGTATTAAGTGAC
CATACGATCATGGGATACTACAGTAAAGTGATCTTCATCTTCAAAGAAC
CATGGTGGCGCGACGCTGGACTTACCGGAATCGTCAACTGTGCGGGTGG
CCCCATAACCTTTACACGGGACACGAGTGTACCCACCGACGACCAATGG
TCTATCACATGCTTCATGGTGGGCAGTCGCGGACGAGCGTGGTCTAAGC
TGTCAAAGGATGATCGATACAGCCAAGTGTGGGAGCAGTTTCGCCGATG
TTTTGAAGAGTTCGTGGAAAACATCCCCGAGCCAGTAAATACCCTGGAG
ATGGAATGGAGTAAAGAGCCTTATTTCCTTGGAGCACCTTGTCCGGCTA
TGATACCCGGCTTACTGACTACTGCCGGGAGTGATCTAGCTGCACCGCA
CGGCAAAGTGCATTTTATCGGAACAGAAACGTCCACAGTGTGGCGTGGG
TACATGGAAGGGGCTATTAGAGCCGGGCAGCGAGGAGGAGCCGAGGTTG
TGACGGCACTGCAGGAAGACTAG.

The amino acid sequence of the amine oxidase is:

(SEQ ID NO: 5)
MSVSNDPTTKLYDAVIVGAGLSGLQAAHSIQAAGFSVCILEATDRMGGK
TLTVKSSEKGYNDLGAAWVNDTNQTEIFKLHQRYGLDGVVQYTCGDDIL
ESGEGVIRKIPYGLPLTGLPKKLLDILRIESSRLDLDDPTSFPGATEVD
NLTFRDFCVEKTGSEDVIHITDAISTALLGLNSNELSALYMLYYFKSGS
GIDNLLSDERDGAQYLRTRQGTQTIARKMADELTQSDIFLGMPVTSINQ
TDADAHCVVQTLDGSSFRCRRVIVSIPTTLYRSVSFHPPLPHAKQVLSD
HTIMGYYSKVIFIFKEPWWRDAGLTGIVNCAGGPITFTRDTSVPTDDQW
SITCFMVGSRGRAWSKLSKDDRYSQVWEQFRRCFEEFVENIPEPVNTLE
MEWSKEPYFLGAPCPAMIPGLLTTAGSDLAAPHGKVHFIGTETSTVWRG
YMEGAIRAGQRGGAEVVTALQED.

The gene encoding the fully sequenced amine oxidase (hereafter referred to as AnFAO) was synthesized by Gene Universal Ltd. and codon optimized (SEQ ID NO: 6) for expression in E. coli. The gene was PCR amplified and ligated into the pET His6 MBP TEV LIC (ligation-independent cloning) vector (Addgene plasmid #29656, a gift from Scott Gradia), placing a Tobacco Etch Virus (TEV) protease cleavage sequence between an N-terminally 6×His-tagged maltose-binding protein (MBP) and the amine oxidase. A sequence verified clone was transformed into E. coli BL21(DE3) and grown at 37° C. until the optical density of the culture at 600 nm (OD₆₀₀) reached 0.5. The temperature was reduced to 16° C. and protein expression was induced with 500 μM isopropyl β-D-1-thiogalactopyranoside (IPTG) and allowed to proceed overnight at 16° C. with shaking. Cells were harvested via centrifugation, re-suspended in 50 mM Tris-HCl (pH 7.4), 500 mM NaCl, 14.3 mM β-mercaptoethanol, 5 mM imidazole, and 5 μM Flavin Adenine Dinucleotide (FAD) (buffer NA), and lysed via sonication.

The lysate supernatant was clarified via centrifugation and subjected to Ni-NTA metal affinity chromatography. Sample was loaded and washed in buffer NA, and batch eluted using buffer NA+250 mM imidazole. Fractions containing AnFAO were pooled and subjected to TEV protease digestion to remove the MBP tag. The protein was digested at a 1:50 ratio of TEV:MBP-AnFAO for 16 hours at 4° C. Following TEV digestion, the sample was brought to 1M (NH₄)₂SO₄ and loaded onto a Phenyl-Sepharose™ HP column equilibrated in 50 mM Tris-HCl (pH 7.4), 1M (NH₄)₂SO₄, 14.3 mM β-mercaptoethanol, and 5 μM FAD (buffer PA).

Protein was eluted using a linear decreasing (NH₄)₂SO₄ gradient (1 M to 0 M) in buffer PA over 10 column volumes. Fractions containing AnFAO were pooled and subjected to gel permeation chromatography using an SEC650 column (Bio-Rad) equilibrated in 50 mM MES buffer (pH 6), 150 mM NaCl, 2 mM dithiothreitol (DTT), and 10 μM FAD (buffer SEC).

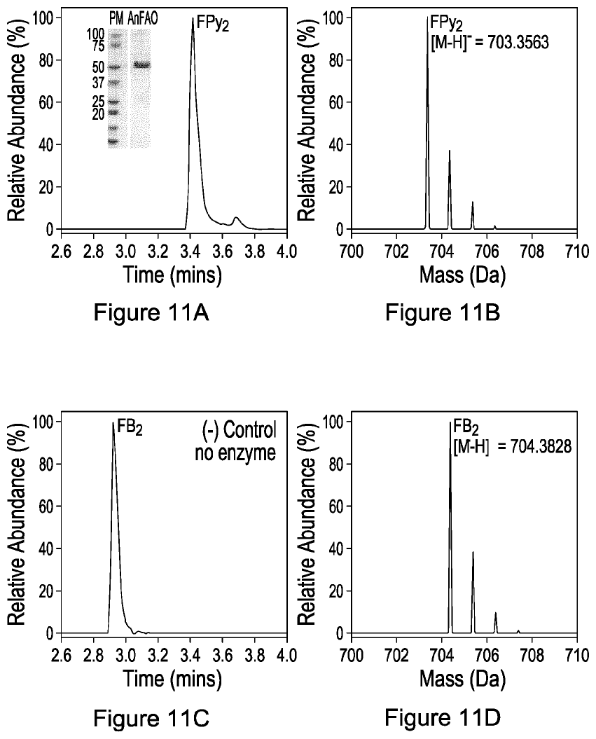

AnFAO was 100% pure as evidenced by SDS-PAGE following this step (FIG. 11A). AnFAO was then diluted to 6 nM in SEC buffer and incubated for 1 h at 37° C. with 1 μM intact FB₂ and 0.1 mg/ml catalase. Full conversion of FB₂ ([M-H]⁻=704.3828) into FPy₂ ([M-H]⁻=703.3563) was observed via reverse-phase LC/MS analysis of samples containing AnFAO as shown in FIGS. 11A and 11B, while no conversion of FB₂ into FPy₂ was observed in the control containing no enzyme as shown in FIGS. 11C and 11D.

### EXAMPLE V

Aspergillus Niger Fumonisin Amine Oxidase In Situ Detoxification of Fumonisin B₂

AnFAO_15309 was also cloned into the EcoRl and Notl restriction sites of pPICZαA and pPICZB vectors to allow for secreted and intracellular expression respectively within the methylotrophic yeast Pichia pastoris. Approximately 10 μg of either pPICZαA-FAO or pPICZB-FAO were transformed into Pichia pastoris strain X33 via electroporation. Transformants were plated onto YPDS (1% Yeast extract, 2% Peptone, 2% Dextrose, 18.2% Sorbitol) agar plates containing 100 μg/ml Zeocin and incubated for 48 hrs at 30° C. Colonies were picked and spotted onto YPDS plates containing 1 mg/ml Zeocin and incubated for 48 hrs at 30° C.

Colonies from each construct were then used to inoculate 10 ml of BMGY liquid media (2.0% Peptone, 1.0% Yeast extract, 100 mM Potassium phosphate pH 6.0, 1.34% Yeast Nitrogen Base (without amino acids), 0.4 μg/mL Biotin, 1.0% Glycerol) containing 100 μg/ml Zeocin and placed in a shaking incubator for 16 hrs at 30° C. Cells were harvested by centrifugation and washed 2× with 10 mL of BMMY liquid media (same as BMGY media except containing 0.5% methanol as carbon source instead of 1.0% glycerol). The cells were then suspended at an OD₆₀₀ of 0.8-1 in 50 mL of BMMY (without zeocin) and transferred to sterile baffled flasks and grown continuously at 30° C. with vigorous shaking (250 rpm).

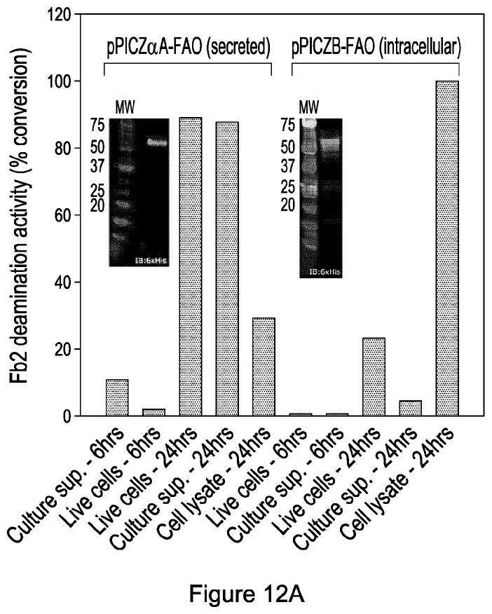

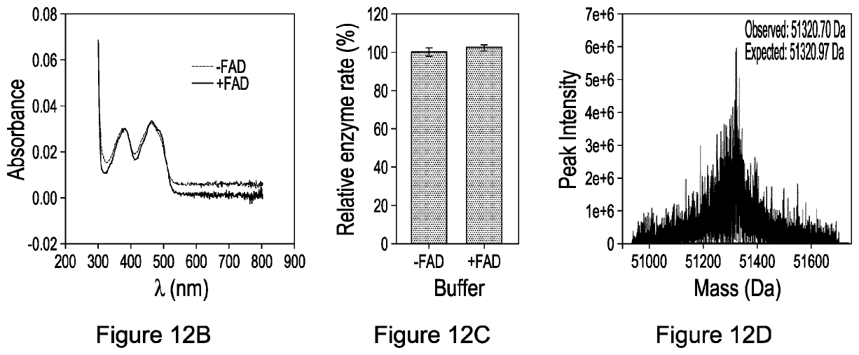

Samples were taken at 6 hrs and 24 hrs post methanol induction, and whole cell suspensions and conditioned media (culture supernatants) were assayed for fumonisin deamination activity via reverse phase LC-MS. After 6 hrs of methanol induction, low levels of fumonisin conversion were observed for both the secreted and intracellular AnFAO clones as shown in FIG. 12A. However, after 24 hrs of induction, both the live cells and the culture supernatant of the pPICZαA-FAO (secreted) clone converted about 90% of the intact FB₂ as shown in FIG. 12A. Only low levels of conversion were observed for the un-lysed live cells expressing the pPICZB-FAO (intracellular). However, upon cell lysis, about 100% conversion of intact FB₂ to FPy₂ was observed as demonstrated in FIG. 12A.

To check for protein expression, culture supernatants from the secreted constructs (pPICZαA-FAO) were concentrated 10-fold using a 1-ml Amicon membrane concentrator (30-kDa cutoff pore size) and subjected to SDS-PAGE followed by immunoblotting using an anti 6×-HIS antibody to detect the recombinant histidine tag. For non-secreted constructs (pPICZB-FAO), cell pellets were lysed in 100 μL phosphate-buffered saline (PBS) (pH 7.4) containing 0.1 mM phenylmethanesulfonyl fluoride (PMSF), and 200 units/ml lyticase using an equal volume of acid washed glass beads and 5 rounds of vortexing and incubation on ice. These were then subject to SDS-PAGE and Western blotting to detect the recombinant protein. A strong signal at the correct MWs was observed for both the secreted and intracellular versions of AnFAO.

### EXAMPLE VI

Characterization of Aspergillus Niger Fumonisin Amine OXIDASE MONOAMINE OXIDASE ACTIVITY

Monoamine oxidases (MAOs; EC 1.4.3.4) are widely distributed throughout nature and oxidize a broad range of nitrogen-containing compounds including primary, secondary, and tertiary amines, polyamines, and amino acids (Fitzpatrick, 2010; Gaweska and Fitzpatrick, 2011). MAOs are flavin-binding enzymes that oxidize amines in a two-step process: FAD is first reduced upon hydride transfer from the substrate generating an imine. The reduced FAD is then oxidized by molecular oxygen, producing H₂O₂ as a by-product, while the imine undergoes spontaneous aqueous hydrolysis to form an aldehyde/ketone. The general reaction mechanism is as follows:

RCH₂NHR′+H₂O+O₂=RCHO+R′NH₂+H₂O₂.

The observation of FAD binding, as shown in Example VII, and measurement of H₂O₂ production via the Amplex™ red assay, as shown in Examples VIII, IX, X, and XI, is consistent with this reaction mechanism for AnFAO. MAOs contain an N-terminal FAD binding domain and a C-terminal substrate binding domain that interact to form a compact globular fold. A multiple sequence alignment indicated residues 18-23 of AnFAO (GAGLSG) constituted the classic G-X-G-X-X-G hexa-peptide motif characteristic of ADP-binding βαβ-folds present within MAOs (Wierenga et al., 1986). Two well-studied MAOs that play key roles in the metabolism of neurotransmitters, MAO-A and MAO-B, contain an additional C-terminal extension following the oxidoreductase domain that allows for embedding within the mitochondrial membrane (Edmondson et al., 2004). This extension was absent in AnFAO. MAO-A and MAO-B also covalently bind FAD via an invariant cysteine residue located near the Flavin ring structure that was absent in AnFAO (Binda et al., 2004a; Binda et al., 2004b). Nevertheless, AnFAO appeared to bind FAD with high affinity as no additional coenzyme was added during purification of the wild-type enzyme from the fungal source, which remained active throughout the course of purification. In addition, AnFAO lacked the ˜120 amino acid N-terminal Reactive Intermediate Deaminase (RID) domain that is found in the fumonisin deaminating amine oxidase from E. spinifera (EsFAO) (Duvick J., 2000). RID proteins catalyze the hydrolysis of reactive imines/enamines, preempting their potential damage within the cell/environment (Niehaus et al., 2015). AnFAO therefore represents a novel class of fungal monoamine oxidases capable of deaminating and detoxifying intact fumonisins.

### EXAMPLE VII

Recombinant Aspergillus Niger Fumonisin Amine Oxidase is a NON-COVALENT FLAVOPROTEIN

Recombinant AnFAO had a distinct yellow color following its isolation from E. coli. Wavelength scans of the purified enzyme dialyzed exhaustively against 20 mM MES (pH 6) and 150 mM NaCl revealed two absorbance maxima at ca. 378 and 462 nm, indicative of the presence of a Flavin Adenine Dinucleotide (FAD) cofactor (Lewis and Escalante-Semerena, 2006; Schilling and Lerch, 1995a) as shown in FIG. 12B. No difference in fumonisin deamination activity was observed when the enzyme was dialyzed in the same buffer with an additional 50 μM FAD as demonstrated in FIG. 12C. The whole mass of AnFAO following denaturation and LC-MS analysis was 51,320.70 Da, which matches the predicted mass of the apo-enzyme (51,320.97 Da) as shown in FIG. 12D. Taken together, these data indicated that 1) recombinant AnFAO was preloaded with FAD following expression in E. coli; 2) the addition of exogenous FAD was not required for fumonisin deamination activity; and 3) AnFAO bound FAD tightly but non-covalently. These results are similar to those observed for MAO-N, another monoamine oxidase produced by A. niger that shows a preference for aliphatic and aromatic amines (Schilling and Lerch, 1995a) but has no activity towards fumonisins (Duvick J., 2000). MAO-N also binds FAD tightly but non-covalently, and sequence analysis indicated that both AnFAO and MAO-N lacked the requisite cysteine residue that would enable 8α-S-Cysteinyl covalent linkages to FAD, as observed in both human MAO-A and MAO-B isoforms. AnFAO also lacked the requisite Histidine or Tyrosine residues for covalent 6α-linkages to the FAD backbone, as observed in other covalent flavoproteins (Heuts et al., 2009).

### EXAMPLE VIII

Characterization of Aspergillus Niger and Aspergillus WELWITSCHIA FUMONISIN AMINE OXIDASE HOMOLOGS

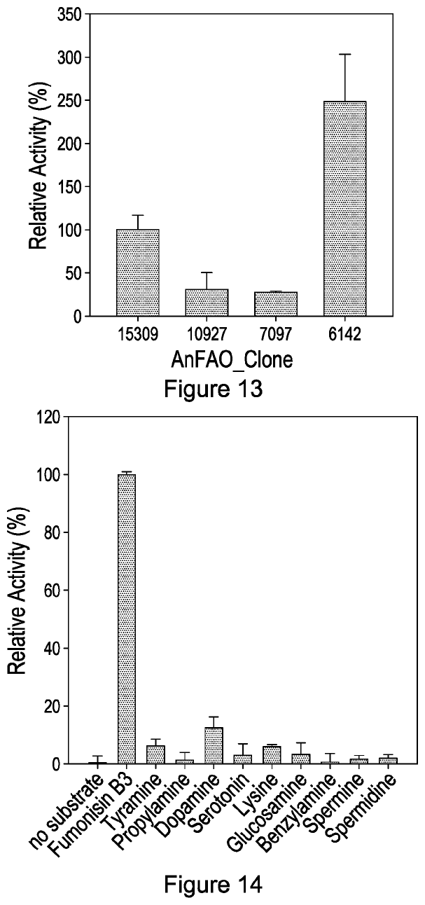

The AnFAO gene was successfully amplified by PCR from 19 of the 23 A. niger and A. welwitschaie strains. Nine of these clones were sequenced in addition to the original AnFAO_15309 clone. Sequencing of each gene demonstrated it is strongly conserved in all strains, with ca. 98% sequence identity from one AnFAO homolog to the next. Clones AnFAO_6142 and AnFAO_10929 shared 100% sequence identity, as did AnFAO_12918 and AnFAO_10954. Seven of the AnFAO homologs were synthesized and cloned into the MBP-TEV vector for recombinant expression in E. coli. AnFAO_5277 and AnFAO_12918 clones could not be purified as they precipitated during purification and appeared to bind FAD poorly as they lacked yellow color following Ni-NTA enrichment (data not shown). Expression and purification of the amine oxidase from A. niger strain CBS513.88 (NCBI accession no. XP_001396491.1) was also attempted, but encountered the same problem as with AnFAO_5277 and AnFAO_12918. Both AnFAO_12918 and the CBS513.88 clone have G to E substitutions at position 445. Based on homology models of AnFAO, this substitution maps to an area predicted to interact with the Flavin ring of FAD. A G445E mutation would disrupt this area and negatively affect the enzyme's ability to bind FAD cofactor. Mutating Gly445 to glutamate in AnFAO_15309 did not yield an enzyme that could be purified, further supporting this hypothesis. The difficulty in purifying AnFAO_5277 is more difficult to rationalize, as it does not contain the G445E substitution. AnFAO clones 6142, 10927, and 7097 alongside AnFAO_15309 were expressed and purified to homogeneity. Their activity was assessed via the Amplex™ red assay, whereby 80 nM enzyme was incubated with 25 μM FB₁, 100 μM Amplex™ red, 1 U/mL HRP, 50 mM HEPES (pH 7) and 150 mM NaCl. AnFAO_6142 was 2.5× more active compared to AnFAO_15309, while clones 10927 and 7097 were less active, with only 31% and 27% activity compared to 15309 as shown in FIG. 13. There are only 3 amino acid substitutions, M46V, R82Q, V267L, between AnFAO_15309 and

AnFAO_6142. AnFAO_15309 (original clone)
(relative activity = 1):
(SEQ ID NO: 5)
MSVSNDPTTKLYDAVIVGAGLSGLQAAHSIQAAGFSVCILEATDRMGGK
TLTVKSSEKGYNDLGAAWVNDTNQTEIFKLHQRYGLDGVVQYTCGDDIL
ESGEGVIRKIPYGLPLTGLPKKLLDILRIESSRLDLDDPTSFPGATEVD
NLTFRDFCVEKTGSEDVIHITDAISTALLGLNSNELSALYMLYYFKSGS
GIDNLLSDERDGAQYLRTRQGTQTIARKMADELTQSDIFLGMPVTSINQ
TDADAHCVVQTLDGSSFRCRRVIVSIPTTLYRSVSFHPPLPHAKQVLSD
HTIMGYYSKVIFIFKEPWWRDAGLTGIVNCAGGPITFTRDTSVPTDDQW
SITCFMVGSRGRAWSKLSKDDRYSQVWEQFRRCFEEFVENIPEPVNTLE
MEWSKEPYFLGAPCPAMIPGLLTTAGSDLAAPHGKVHFIGTETSTVWRG
YMEGAIRAGQRGGAEVVTALQED.
AnFAO_10927 (relative activity = 0.31):
(SEQ ID NO: 27)
MSVSNDPTTKLYDAVIVGAGLSGLQAAHSIQAAGFSVCILEATDRVGGK
TLTVKSSEKGYNDLGAAWVNDTNQTEIFKLHQRYGLDGVVQYTCGDDIL
ESGEGVIRKIPYGLPLTGLPKKLLDILRIESSRLDLDDPTSFPGATEVD
NLTFRDFCVEKTGSEDVIHITDAISTALLGLNSNELSALYMLYYFKSGS
GIDNLLSDERDGAQYLRTRQGTQTIARKMADELSQSDIFLGMPVTSINQ
TDADAHCVVKTLDGSSFRCRRVIVSIPTTLYRSVSFHPPLPHAKQVLSD
HTIMGYYSKVIFIFKEPWWRDAGLTGIVDCAGGPITFTRDTSVPTDDQW
SITCFMVGSRGRAWSKLSKDDRYSQVWEQFRRFFEEFVENIPEPANTLE
MEWSKEPYFLGAPCPAMIPGLLTTAGSDLAAPHGKVHFIGTETSTVWRG
YMEGAIRAGQRGGAEVVTALQED.
AnFAO_6142 (identical to AnFAO_10929)
(relative activity = 2.5):
(SEQ ID NO: 28)
MSVSNDPTTKLYDAVIVGAGLSGLQAAHSIQAAGFSVCILEATDRVGGK
TLTVKSSEKGYNDLGAAWVNDTNQTEIFKLHQQYGLDGVVQYTCGDDIL
ESGEGVIRKIPYGLPLTGLPKKLLDILRIESSRLDLDDPTSFPGATEVD
NLTFRDFCVEKTGSEDVIHITDAISTALLGLNSNELSALYMLYYFKSGS
GIDNLLSDERDGAQYLRTRQGTQTIARKMADELTQSDIFLGMPVTSINQ
TDADAHCVVQTLDGSSFRCRRLIVSIPTTLYRSVSFHPPLPHAKQVLSD
HTIMGYYSKVIFIFKEPWWRDAGLTGIVNCAGGPITFTRDTSVPTDDQW
SITCFMVGSRGRAWSKLSKDDRYSQVWEQFRRCFEEFVENIPEPVNTLE
MEWSKEPYFLGAPCPAMIPGLLTTAGSDLAAPHGKVHFIGTETSTVWRG
YMEGAIRAGQRGGAEVVTALQED.
AnFAO_7097 (relative activity = 0.27):
(SEQ ID NO: 29)
MSVSNDPTTKLYDAVIVGAGLSGLQAAHSIQAAGFSVCILEATDRVGGK
TLTVKSSEKGYNDLGAAWVNDTNQTEIFKLHQQYGLDGVVQYTCGDDIL
ESGEGVIRKIPYGLPLTGLPKKLLDILRIESSRLDLDDPTSFPGATEVD
NLTFRDFCVEKTGSEDVIHITDAISTALLGLNSNELSALYMLYYFKSGS
GIDNLLSDERDGAQYLRTRQGTQTIARKMADELTQSDIFLGMPVTSINQ
TDADAHCVVQTLDGSSFRCRRVIVSIPTTLYRSVSFHPPLPHAKQVLSD
HTIMGYYSKVIFIFKEPWWRDAGLTGIVDCAGGPITFTRDTSVPTDDQW
SITCFMVGSRGRAWSKLSKDDRYSQVWEQFRRCLEGFVENIPEPANTLE
MEWSKEPYFLGAPCPAMIPGLLTTTGSDLAAPHGKVHFIGTETSTVWRG
YMEGAIRAGQRGGAEVVTALQED.
AnFAO_5277-could not purify:
(SEQ ID NO: 30)
MSVSNDPTTKLYDAVIVGAGLSGLQAAHSIQAAGFSVCILEATDRVGGK
TLTVKSSEKGYNDLGAAWVNDTNQTEIFKLHQQYGLDGVVQYTCGDDIL
ESGEGVIRKIPYGLPLTGLPKKLLDILRIESSRLDLDDPTSFPGATEVD
NLTFRDFCVEKTGSEDVIHITDAISTALLGLNSNELSALYMLYYFKSGS
GIDNLLSDERDGAQYLRTRQGTQTIARKMADELSQSDIFLGMPVTSINQ
TDADAHCVVQTLDGSSFRCRRVIVSIPTTLYRSVSLHPPLPHAKQVLSD
HTIMGYYSKVIFIFKEPWWRDAGLTGIVDCAGGPITFTRDTSVPTDDQW
SITCFMVGSRGRAWSKLSKDDRYSQVWEQFRRCFEEFVENIPEPVNTLE
MEWSKEPYFLGAPCPAMIPGLLTTAGSDLAAPHGKVHFIGTETSTVWRG
YMEGAIRAGQRGGAEVVTALQED.
AnFAO_12918-could not purify (same sequence as
AnFAO_10954):
(SEQ ID NO: 31)
MSVSNDPTTKLYDAVIVGAGLSGLQAAHSIQAAGFSVCILEATDRVGGK
TLTVKSSEKGYNDLGAAWVNDTNQTEIFKLHQRYGLDGVVQYPCGDDIL
ESGEGVIRKIPYGLPLTGLPKKLLDILRIESSRLDLDDPMSFPGATEVD
NLTFRDFCVEKTGSEDVIHITDAISTALLGLNSNELSALYMLYYFKSGS
GIDNLLSDERDGAQYLRTRQGTQTIARKMADELSQSDIFLGMPVTSINQ
TDADAHCVVQTLDGSSFRCRRVIVSIPTTLYRSVSFHPPLPHAKQVLSD
HTIMGYYSKVIFIFKEPWWRDAGLTGIVDCAGGPITFTRDTSVPTDDQW
SITCFMVGSRGRAWSKLSKDDRYSQVWEQFRRCFEDFVENIPEPANTLE
MEWSKEPYFLGAPCPAMIPGLLTTAGSDVAAPHGKVHFIGTETSTVWRG
YMEEAIRAGQRGGAEVVTALQED.
CBS_513.88 (XP_001396491.1)-could not purify:
(SEQ ID NO: 32)
MSVSNDPTTKLYDAVIVGAGLSGLQAAHSIQAAGFSVCILEATDRVGGK
TLTVKSSEKGYNDLGAAWVNDTNQTEIFKLHQRYGLDGVVQYPCGDDIL
ESGEGVIRKIPYGLPLTGLPKKLLDILRIESSRLDLDDPMSFPGATEVD
NLTFRDFCVEKTGSEDVIHITDAISTALLGLNSNELSALYMLYYFKSGS
GIDNLLSDERDGAQYLRTRQGTQTIARKMADELSQSDIFLGMPVISINQ
TDADAHCVVQTLDGSSFRCRRVIVSIPTTLYRSVSFHPPLPHAKQVLSD
HTIMGYYSKVIFIFKEPWWRDAGLTGIVDCAGGPITFTRDTSVPTDDQW
SITCFMVGSRGRAWSKLSKDDRYSQVWEQFRRCFEDFVENIPEPANTLE
MEWSKEPYFLGAPCPAMIPGLLTTAGSDVAAPHGKVHFIGTETSTVWRG
YMEEAIRAGQRGGAEVVTALQED.

### EXAMPLE IX

Kinetic Analysis and Substrate Specificity

To test the activity of AnFAO_15309 towards various amine containing substrates, the enzyme was assayed in triplicate at a concentration of 20 nM in 50 mM HEPES pH 7.0, 150 mM NaCl, 100 μM Amplex™ red, 1 U/ml horseradish peroxidase and 50 μM substrate. Absorbance was monitored at 571 nm every 10 mins for 1 hr. A standard curve consisting of 0-10 μM H₂O₂ was included to determine the absorbance for every μmol of H₂O₂ generated by the reaction. Relative to FB₃ (100% activity), AnFAO_15309 displayed little activity towards aromatic and short chain amine-containing substrates including propylamine (5.6%), benzylamine (1.4%), dopamine (9.7%), serotonin (3.7%), tyramine (5.8%), lysine (6.3%), and glucosamine (1.2%). It also displayed background levels of activity towards the polyamines spermine (1.9%) and spermidine (1.4%) as shown in FIG. 14.

Kinetic analysis of recombinant AnFAO_15309 indicates the enzyme was 3.2-fold more efficient at deaminating FB₂ compared to FB₁. The increased performance results from an ˜2-fold stronger affinity (K_{M}=194.7 μM FB₂ vs. 390.6 μM FB₁) and ˜1.6-fold increase in catalytic efficiency (k_{cat}=13.7 min⁻¹ FB₂ vs. 8.7 min⁻¹ FB₁) (Table 1). Interestingly, AnFAO displayed significant activity towards additional long-chain aliphatic amino alcohols, including hydrolyzed FB₁ and sphinganine (Table 1).

| | FB₁ | FB₂ | hFB₁ | sphinganine |
|---|---|---|---|---|
| k_{cat} (min⁻¹) | 8.7 | 13.7 | 17.9 | 36.7 |
| K_{M} (μM) | 390.6 | 194.7 | 55.2 | 31.4 |
| k_{cat}/K_{M} (M⁻¹s⁻¹) | 3.7E+02 | 1.2E+03 | 5.4E+03 | 1.9E+04 |
| fold change | 0.3 | 1.0 | 4.6 | 16.6 |

Compared to FB₂, AnFAO was 4.6-fold more efficient at deaminating hydrolyzed FB₁, and 16.6-fold more efficient at deaminating sphinganine. The majority of the increased performance derived from significant increases in substrate affinity (K_{M}=55.2 μM for hydrolyzed FB₁ and 31.4 μM for sphinganine), with relatively smaller increases in catalytic turnover (k_{cat}=17.9 min⁻¹ for hydrolyzed FB1 and 36.7 min⁻¹ for sphinganine). Hydrolyzed FB₁ was produced by incubating 10 mg of FB₁ in 2M KOH overnight at room temperature. The resulting mixture was twice extracted with equal volumes of ethyl acetate, dried, and re-suspended in reaction buffer.

### EXAMPLE X

Enzymatic Properties

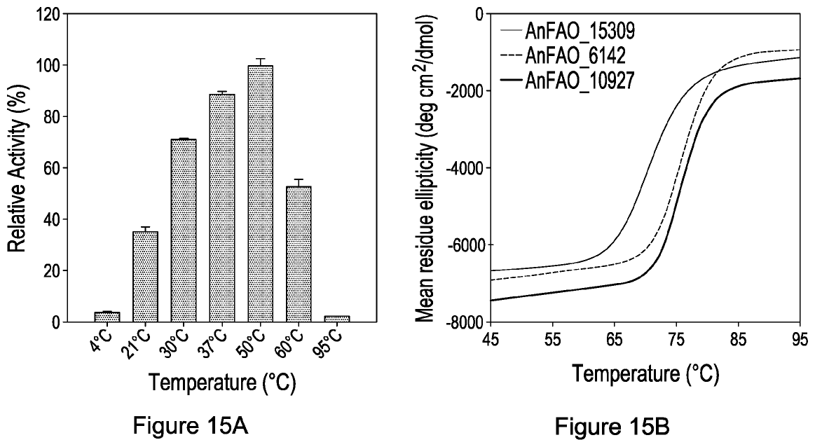

To determine the effect of temperature on fumonisin deamination activity, 6 nM AnFAO_15309 was incubated at 4°, 21°, 30°, 37°, 50°, 60°, and 95° C. in the presence of 2 μM FB₂. Activity was measured via reverse-phase LC/MS analysis. AnFAO deaminated FB₂ optimally at 50° C., while robust activity was maintained across a broad temperature spectrum, with ca. 35% of maximal activity displayed at 21° C., and 52% of maximal activity remaining at 60° C. as shown in FIG. 15A. No activity was observed at 95° C. AnFAO_15309 had a melting temperature of 71° C., while AnFAO clones 6142 and 10927 both displayed melting temperatures of 76° C., ca. 5° C. higher than AnFAO_15309 as determined using a Jasco J-810 circular dichroism spectropolarimeter as shown in FIG. 15B. All proteins were scanned at a concentration of 0.3 mg/ml in buffer containing 20 mM Tris-HCl (pH 7.4), 50 mM NaCl and 0.75 mM DTT. Thermal denaturation was monitored at 222 nM with a temperature scan ranging from 20 to 95° C., increasing by 1° C. per minute with data collection every 30 seconds. Baseline corrected data were converted from millidegrees to mean residue ellipticity, and thermal denaturation curves were produced by nonlinear regression in Graphpad Prism.

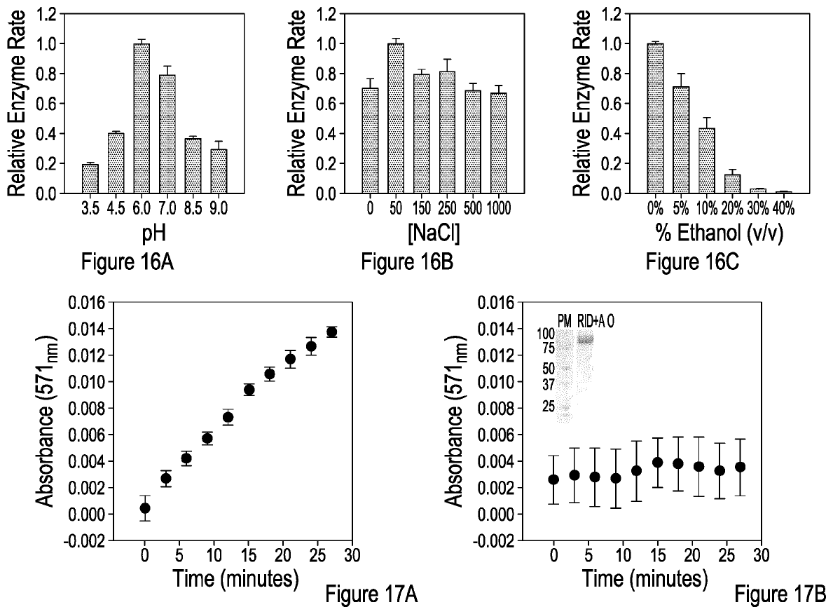

To measure the optimal pH for fumonisin deamination activity, AnFAO_15309 was diluted to 300 nM in 150 mM NaCl and various buffers at the desired pH (Citrate pH 3.5, Citrate pH 4.5, MES pH 6, HEPES pH 7, Tris-HCl pH 8.5, and Pyrophosphate pH 9). 98 μL of this solution was added to 2 μL of a stock of FB₂ in a microfuge tube (2.5 mM) and incubated at 30° C. for 30 mins. At the end of the incubation time, 20 μL of the reaction was diluted with 80 μL of 50 mM HEPES pH 7.0, 150 mM NaCl and 100 μL of this was added in triplicate to wells of a 96-well microplate. 100 μL of Amplex™ red reaction buffer consisting of 50 mM HEPES pH 7.0, 150 mM NaCl, 200 μM Amplex™ red, 2 U/ml horseradish peroxidase was immediately added to each well and absorbance was measured at 571 nm. Rates were calculated based on an H₂O₂ standard curve and the incubation time taking into account the dilution factors. AnFAO displayed broad activity across all pH's tested as shown in FIG. 16A. Optimal activity occurred at pH 6, with ca. 20% activity remaining at pH 3.5, and ca. 30% activity remaining at pH 9.0. To test the effect of increasing salt concentrations, AnFAO_15309 was diluted to 300 nM in 50 mM HEPES pH 7.0 containing 0 to 1 M NaCl. 100 μL of protein sample was added to wells in triplicate in a 96-well microplate. 100 μL of Amplex™ red reaction buffer consisting of 50 mM HEPES pH 7.0, 200 μM Amplex™ red, 2 U/ml horseradish peroxidase and 50 μM FB₂ was then added to each well and absorbance was monitored at 571 nm every 10 mins for 1 hr. A standard curve consisting of 0-10 μM H₂O₂ was included to determine the absorbance for every pmol of H₂O₂ generated by the reaction. AnFAO was relatively insensitive to changes in NaCl concentrations, with optimal activity occurring at 50 mM NaCl, while 66% of maximum activity remained at 1 M NaCl and 70% of maximum activity remaining 0 mM NaCl as demonstrated in FIG. 16B. AnFAO_15309 also displayed robust activity in the presence of increasing amounts of ethanol as displayed in FIG. 16C. 10 nM AnFAO_15309 was incubated for 30 minutes at 37° C. with 1 μM FB₂ in 50 mM HEPES pH 7.0, 150 mM NaCl and in the presence of increasing amounts of EtOH, and samples were then analyzed via reverse phase LC/MS analysis tracking % conversion to the deaminated form. Deamination activity gradually decreased in the presence of increasing ethanol, with 71% activity remaining at 5% EtOH, 43% activity remaining at 10% EtOH, and 12% activity remaining at 20% EtOH. Negligible amounts of deamination activity were observed at 30% and 40% EtOH.

### EXAMPLE XI

Characterization of Anfao Homologs

BLASTp searches of Aspergillus niger genomes indicated the presence of multiple putative amine oxidases. The next closest homolog to AnFAO, based on amino acid conservation, was a 597 amino acid enzyme that contained a conserved Reactive Intermediate Deaminase (RID) domain at its N-terminus followed by an amine oxidase domain at its C-terminus that was ˜40% identical and ˜56% similar to AnFAO. RID domains enhance the hydrolysis of chemically reactive imines, preventing potential side reactions that form damaged compounds toxic to the cell (Niehaus et al., 2015; Niehaus et al., 2014). The architecture of the gene was most similar to the fumonisin amine oxidase originally identified in E. spinifera that also contained an N-terminal RID domain fused upstream of a C-terminal amine oxidase domain (Duvick J., 2000; Duvick J., 1998). The RID+AO gene (SEQ ID NO: 33) was PCR amplified out of A. niger strain 15309 using PCR primers AORIDF1: 5′-AAGTCAACACTTCCCCGCACG-3′ (SEQ ID NO: 34) and AORIDR1: 5′-TATAGCACGAGTGCCTCGGAA-3′ (SEQ ID NO: 35) and sequenced. The enzyme (SEQ ID NO: 36) was ca. 98% identical at the amino acid level to its equivalent RID+AO homologs (e.g. NCBI accession numbers EHA25009.1, GCB21444.1) within other sequenced A. niger strains. The gene was synthesized and codon optimized for bacterial expression with an N-terminal Glutathione-S-transferase tag. The tagged protein was purified to homogeneity in a similar manner as AnFAO following recombinant expression in E. coli. The purified recombinant enzyme had a distinct yellow color upon isolation (data not shown), indicating FAD binding similar to AnFAO. Both AnFAO and the RID+AO enzymes were assayed for fumonisin deamination activity in triplicate using the Amplex™ red assay as shown in FIGS. 17A and 17B, respectively. The final concentration of reagents and enzymes in the assay were 100 μM Amplex™ red, 1 U/mL HRP, 25 μM FB₁, and 80 nM of each enzyme in 50 mM HEPES (pH 7) and 150 mM NaCl. Under these conditions, the RID+AO enzyme displayed no activity towards fumonisins as demonstrated in FIG. 17B. These data indicated that fumonisin deamination activity was not a general property of amine oxidases from A. niger. This data was further bolstered by the fact MAO-N, another well characterized monoamine oxidase produced by A. niger also did not deaminate fumonisins (Duvick J., 2000; Schilling and Lerch, 1995a; Schilling and Lerch, 1995b). It was likely the RID+AO enzyme from A. niger preferentially deaminates non-fumonisin amine containing compounds.

### EXAMPLE XII

Expression in Saccharomyces Cerevisiae

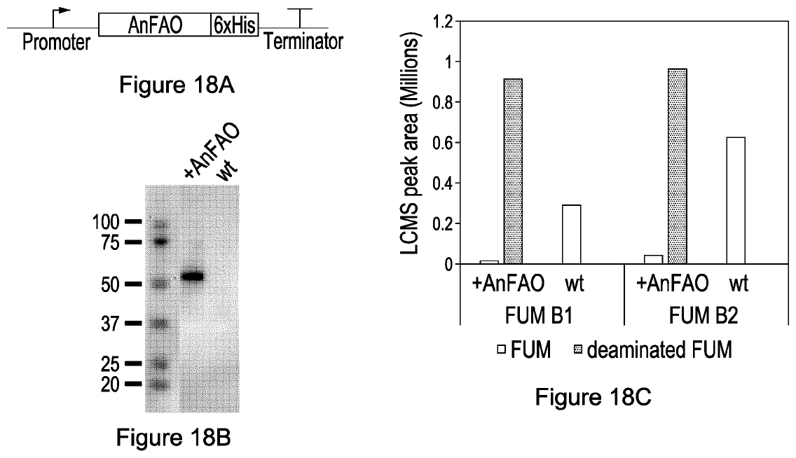

AnFAO_15309 was codon-optimized (SEQ ID NO: 37) and integrated into the genome of Saccharomyces cerevisiae. To verify the expression, a His-tag was fused to the C-terminus of AnFAO as depicted in FIG. 18A. The cell pellet from the resulting AnFAO-expressing strain was lysed via bead beating in TBS buffer (Tris-Buffered Saline at pH 7.4 with protease inhibitor), and the soluble fraction was probed with an anti-His tag antibody. The western blot showed a prominent band slightly above 50 kDa, which corresponded to the predicted molecular weight as shown in FIG. 18B. To examine the activity of the S. cerevisiae-expressed AnFAO, the soluble fraction was incubated with 100 ppm of FB₁ or FB₂. The sample was incubated at 37° C. for 12 hours, followed by 99° C. for 15 min to stop the reaction. LC-MS was used to analyze FB₁/B₂ and the corresponding deaminated products. Deaminated FB₁ or FB₂ were detected in the samples with cell lysate from the AnFAO-expressing strain while only intact fumonisins were detected from the wild-type strain as demonstrated in FIG. 18C. The LC-MS result indicated that S. cerevisiae-expressed AnFAO deaminated FB₁ and FB₂.

### EXAMPLE XIII

Anfao Deaminates Fumonisins in a Complex Matrix

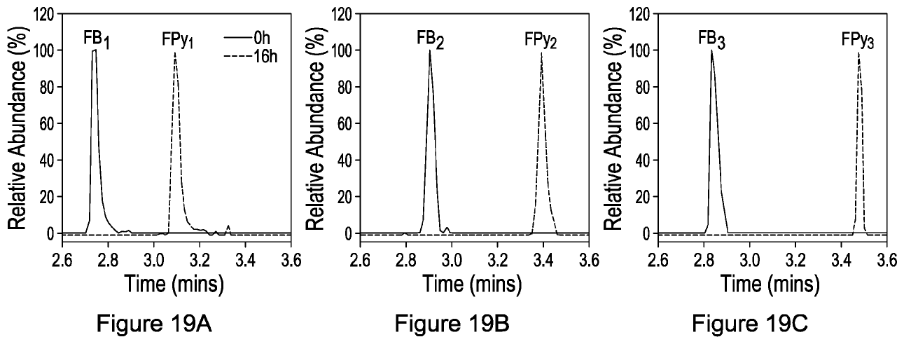

Low level fumonisins in corn quality control material (150 mgs) from Romer labs (initial fumonisin levels (μg/kg): 667±78 FB₁ 156±21 FB₂, and 89±22 FB₃) was re-suspended in 500 μls of milli-Q water. Purified recombinant AnFAO_15309 was diluted to 1 μM final concentration in the mixture, which was then incubated at room temperature for 16 h with shaking. Following incubation, 700 μls of extraction solution (78% acetonitrile, 2% ethyl acetate) was added to each sample and incubated at 37° C. with shaking for 45 minutes. This mixture was then centrifuged at 20,000×g and 400 μls of the cleared supernatant was mixed with an equal volume of 50% methanol. Samples were then analyzed for fumonisin contamination by reverse phase HPLC-MS as previously described. Following treatment with AnFAO_15309, no intact fumonisins remained, and they were all converted into oxidized counterparts as shown in FIG. 19. These results indicated that purified recombinant AnFAO_15309 was capable of completely deaminating fumonisins in a complex matrix contaminated with multiple fumonisin chemotypes including FB₁, FB₂, and FB₃.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Binda, C., Hubalek, F., Li, M., Edmondson, D. E. and Mattevi, A. (2004a) Crystal structure of human monoamine oxidase B, a drug target enzyme monotopically inserted into the mitochondrial outer membrane. FEBS letters 564, 225-228.

Binda, C., Hubalek, F., Li, M., Herzig, Y., Sterling, J., Edmondson, D. E. and Mattevi, A. (2004b) Crystal structures of monoamine oxidase B in complex with four inhibitors of the N-propargylaminoindan class. Journal of medicinal chemistry 47, 1767-1774.

Burgess, K. M., Renaud, J. B., McDowell, T. and Sumarah, M. W. (2016) Mechanistic Insight into the Biosynthesis and Detoxification of Fumonisin Mycotoxins. ACS chemical biology 11, 2618-2625.

Canadian Food Inspection Agency (2017) Multi-Mycotoxin Analysis in Selected Foods.

Chatterjee R., D. J., English J. (2003) AP1 Amine Oxidase Variants. United States: Maxygen, Inc.

Duvick, J., Rood T., Maddox J, Gilliam J (1998) Detoxification of mycotoxins in planta as a strategy for improving grain quality and disease resistance: identification of fumonisin-degrading microbes from maize. Molecular Genetics of Host-Specific Toxins in Plant Disease, 369-381.

Duvick J., G. J., and Maddox J. R (2000) Amino Polyol amine oxidase polynucleotides and related polypeptides and methods of use. United States: Pioneer Hi-Bred.

Duvick J., R. T., Maddox J. R, and Wang X. (1998) Fumonisin Detoxification Compositions and Methods. United States: Pioneer Hi-Bred International.

Edmondson, D. E., Mattevi, A., Binda, C., Li, M. and Hubalek, F. (2004) Structure and mechanism of monoamine oxidase. Current medicinal chemistry 11, 1983-1993.

Fitzpatrick, P. F. (2010) Oxidation of amines by flavoproteins. Archives of biochemistry and biophysics 493, 13-25.

Gaweska, H. and Fitzpatrick, P. F. (2011) Structures and Mechanism of the Monoamine Oxidase Family. Biomol Concepts 2, 365-377.

Gelderblom, W. C., Kriek, N. P., Marasas, W. F. and Thiel, P. G. (1991) Toxicity and carcinogenicity of the Fusarium moniliforme metabolite, fumonisin B1, in rats. Carcinogenesis 12, 1247-1251.

Gelderblom, W. C., Semple, E., Marasas, W. F. and Farber, E. (1992) The cancer-initiating potential of the fumonisin B mycotoxins. Carcinogenesis 13, 433-437.

Grenier, B., Schwartz-Zimmermann, H. E., Gruber-Dorninger, C., Dohnal, I., Aleschko, M., Schatzmayr, G., Moll, W. D. and Applegate, T. J. (2017) Enzymatic hydrolysis of fumonisins in the gastrointestinal tract of broiler chickens. Poult Sci.

Harrison, L. R., Colvin, B. M., Greene, J. T., Newman, L. E. and Cole, J. R., Jr. (1990) Pulmonary edema and hydrothorax in swine produced by fumonisin B1, a toxic metabolite of Fusarium moniliforme. Journal of veterinary diagnostic investigation: official publication of the American Association of Veterinary Laboratory Diagnosticians, Inc 2, 217-221.

Hartinger, D., Heinl, S., Schwartz, H. E., Grabherr, R., Schatzmayr, G., Haltrich, D. and Moll, W. D. (2010) Enhancement of solubility in Escherichia coli and purification of an aminotransferase from Sphingopyxis sp. MTA144 for deamination of hydrolyzed fumonisin B(1). Microbial cell factories 9, 62.

Hartinger, D., Schwartz, H., Hametner, C., Schatzmayr, G., Haltrich, D. and Moll, W. D. (2011) Enzyme characteristics of aminotransferase FumI of Sphingopyxis sp. MTA144 for deamination of hydrolyzed fumonisin B(1). Applied microbiology and biotechnology 91, 757-768.

Heinl, S., Hartinger, D., Thamhesl, M., Schatzmayr, G., Moll, W. D. and Grabherr, R. (2011) An aminotransferase from bacterium ATCC 55552 deaminates hydrolyzed fumonisin B(1). Biodegradation 22, 25-30.

Hein!, S., Hartinger, D., Thamhesl, M., Vekiru, E., Krska, R., Schatzmayr, G., Moll, W. D. and Grabherr, R. (2010) Degradation of fumonisin B1 by the consecutive action of two bacterial enzymes. Journal of biotechnology 145, 120-129.

Irvine, G. W., Heinlein, L., Renaud, J. B., Sumarah, M. W. and Stillman, M. J. (2017) Formation of oxidative and non-oxidative dimers in metallothioneins: Implications for charge-state analysis for structural determination. Rapid communications in mass spectrometry: RCM 31, 2118-2124.

Marasas, W. F., Kellerman, T. S., Gelderblom, W. C., Coetzer, J. A., Thiel, P. G. and van der Lugt, J. J. (1988) Leukoencephalomalacia in a horse induced by fumonisin B1 isolated from Fusarium moniliforme. The Onderstepoort journal of veterinary research 55, 197-203.

Merrill, A. H., Jr., Sullards, M. C., Wang, E., Voss, K. A. and Riley, R. T. (2001) Sphingolipid metabolism: roles in signal transduction and disruption by fumonisins. Environ Health Perspect 109 Suppl 2, 283-289.

Miller, J. D. (2001) Factors that affect the occurrence of fumonisin. In: Environmental Health Perspectives pp. 321-324.

Missmer, S. A., Suarez, L., Felkner, M., Wang, E., Merrill, A. H., Jr., Rothman, K. J. and Hendricks, K. A. (2006) Exposure to fumonisins and the occurrence of neural tube defects along the Texas-Mexico border. Environ Health Perspect 114, 237-241.

Niehaus, T. D., Gerdes, S., Hodge-Hanson, K., Zhukov, A., Cooper, A. J., ElBadawi-Sidhu, M., Fiehn, O., Downs, D. M. and Hanson, A. D. (2015) Genomic and experimental evidence for multiple metabolic functions in the RidA/YjgF/YER057c/UK114 (Rid) protein family. BMC genomics 16, 382.

Norred, W. P., Riley, R. T., Meredith, F. I., Poling, S. M. and Plattner, R. D. (2001) Instability of N-acetylated fumonisin B1 (FA1) and the impact on inhibition of ceramide synthase in rat liver slices. Food Chem Toxicol 39, 1071-1078.

Qi, T. F., Renaud, J. B., McDowell, T., Seifert, K. A., Yeung, K. K. and Sumarah, M. W. (2016) Diversity of Mycotoxin-Producing Black Aspergilli in Canadian Vineyards. Journal of agricultural and food chemistry 64, 1583-1589.

Renaud, J. B., Kelman, M. J., Qi, T. F., Seifert, K. A. and Sumarah, M. W. (2015) Product ion filtering with rapid polarity switching for the detection of all fumonisins and AAL-toxins. Rapid communications in mass spectrometry: RCM 29, 2131-2139.

Rheeder, J. P., W. F. O. Marasas, P. G. Thiel, E. W. Sydenham, G. S. Sherphard., D. J. Van Schalkwyk (1992) Fusarium moniliforme and fumonisins in relation to human esophageal cancer in Transkei. Phytopathology 82, 353-357.

Riley, R. T., Enongene, E., Voss, K. A., Norred, W. P., Meredith, F. I., Sharma, R. P., Spitsbergen, J., Williams, D. E., Carlson, D. B. and Merrill, A. H., Jr. (2001) Sphingolipid perturbations as mechanisms for fumonisin carcinogenesis. Environ Health Perspect 109 Suppl 2, 301-308.

Täubel, M. (2005) Isolierung and Charakterisierung von Mikroorganismen zur biologischen Inaktivierung von Fumonisinen. Doctoral thesis. University of Natural Resources and Applied Life Sciences. Vienna, Austria.

Vanhoutte, I., Audenaert, K. and De Gelder, L. (2016) Biodegradation of Mycotoxins: Tales from Known and Unexplored Worlds. Front Microbiol 7, 561.

Vaudel, M., Barsnes, H., Berven, F. S., Sickmann, A. and Martens, L. (2011) SearchGUl: An open-source graphical user interface for simultaneous OMSSA and X!Tandem searches. Proteomics 11, 996-999.

Vaudel, M., Burkhart, J. M., Zahedi, R. P., Oveland, E., Berven, F. S., Sickmann, A., Martens, L. and Barsnes, H. (2015) PeptideShaker enables reanalysis of MS-derived proteomics data sets. Nature biotechnology 33, 22-24.

Voss, K. A., Howard, P. C., Riley, R. T., Sharma, R. P., Bucci, T. J. and Lorentzen, R. J. (2002) Carcinogenicity and mechanism of action of fumonisin B1: a mycotoxin produced by Fusarium moniliforme (=F. verticillioides). Cancer Detect Prev 26, 1-9.

Wierenga, R. K., Terpstra, P. and Hol, W. G. (1986) Prediction of the occurrence of the ADP-binding beta alpha beta-fold in proteins, using an amino acid sequence fingerprint. Journal of molecular biology 187, 101-107.

Wu F.; Bhatnagar D.; Bui-Klimke, T. C., I.; Hellmich, R. L. (2011) Climate change impacts on mycotoxin risks in US maize. World Mycotoxin Journal 4, 79-93.

What is claimed is:

1. A recombinant microbial host cell expressing an heterologous polypeptide having fumonisin amine oxidase activity, the recombinant microbial host cell comprising an heterologous nucleic acid molecule encoding the heterologous polypeptide having fumonisin amine oxidase activity, wherein the heterologous polypeptide has the amino acid sequence of SEQ ID NO: 5, SEQ ID NO: 27, SEQ ID NO: 28, or SEQ ID NO: 29, is a variant of the amino acid sequence of SEQ ID NO: 5, SEQ ID NO: 27, SEQ ID NO: 28 or SEQ ID NO: 29 having fumonisin amine oxidase activity or is a fragment of the amino acid sequence of SEQ ID NO: 5, SEQ ID NO: 27, SEQ ID NO: 28 or SEQ ID NO: 29 having fumonisin amine oxidase activity.

2. The recombinant microbial host cell of claim 1, wherein the variant or the fragment has at least 70%, 80%, 90% or 95% identity with respect to the amino acid sequence of SEQ ID NO: 5, SEQ ID NO: 27, SEQ ID NO: 28 or SEQ ID NO: 29.

3. The recombinant microbial host cell of claim 1 or 2, wherein the heterologous nucleic acid molecule allows the expression of an intracellular form of the heterologous polypeptide having fumonisin amine oxidase activity.

4. The recombinant microbial host cell of claim 1 or 2, wherein the heterologous nucleic acid molecule allows the expression of a secreted form of the heterologous polypeptide having fumonisin amine oxidase activity.

5. The recombinant microbial host cell of claim 4, wherein the heterologous nucleic acid molecule is operatively associated with a further nucleic acid molecule encoding a signal sequence peptide.

6. The recombinant microbial host cell of claim 1 or 2, wherein the heterologous nucleic acid molecule allows the expression of a membrane-associated form of the polypeptide having heterologous fumonisin amine oxidase activity.

7. The recombinant microbial host cell of claim 6, wherein the membrane-associated form of the heterologous polypeptide having fumonisin amine oxidase activity is a tethered form of the heterologous polypeptide having fumonisin amine oxidase activity.

8. The recombinant microbial host cell of any one of claims 1 to 7 being a yeast host cell.

9. The recombinant microbial host cell of claim 8 being from the genus Saccharomyces.

10. The recombinant microbial host cell of claim 9 being from the species Saccharomyces cerevisiae.

11. The recombinant microbial host cell of claim 8 being from the genus Pichia.

12. The recombinant microbial host cell of claim 11 being from the species Pichia pastoris.

13. The recombinant microbial host cell of any one of claims 1 to 7 being a fungal host cell.

14. The recombinant microbial host cell of claim 13 being from the genus Aspergillus.

15. The recombinant microbial host cell of claim 13 being from the genus Trichoderma.

16. The recombinant microbial host cell of any one of claims 1 to 7 being a bacterial host cell.

17. The recombinant microbial host cell of claim 16 being from the genus Bacillus.

18. The recombinant microbial host cell of claim 17 being from the species Bacillus subtilis.

19. The recombinant microbial host cell of claim 16 being from the genus Escherichia.

20. The recombinant microbial host cell of claim 19 being from the species Escherichia coli.

21. A microbial composition comprising (i) the heterologous polypeptide having fumonisin amine oxidase activity defined in any one of claims 1 to 20 and (ii) the recombinant microbial host cell of any one of claims 1 to 20 or at least one component from the recombinant microbial host cell of any one of claims 1 to 20.

22. The microbial composition of claim 21 comprising the recombinant microbial host cell.

23. The microbial composition of claim 21 comprising the at least one component from the recombinant microbial host cell.

24. The microbial composition of claim 23, wherein the at least one component comprises or is from a lysed recombinant microbial host cell.

25. A process for making an isolated, synthetic or recombinant polypeptide having heterologous fumonisin amine oxidase activity, the process comprising:
a) propagating the recombinant microbial host cell of any one of claims 1 to 20 to obtain a propagated recombinant microbial host cell and the heterologous fumonisin amine oxidase;
b) dissociating the propagated microbial host cell from the heterologous polypeptide having fumonisin amine oxidase activity to obtain a dissociated fraction enriched in the heterologous polypeptide having the fumonisin amine oxidase activity or lysing the propagated microbial host cell to obtained a lysed fraction;
c) optionally drying the dissociated or lysed microbial host cell to obtain a dried fraction; and
d) substantially purifying the heterologous polypeptide having fumonisin amine oxidase activity from the dissociated, lysed or dried fraction to provide the isolated, synthetic or recombinant heterologous polypeptide having fumonisin amine oxidase activity.

26. An isolated, synthetic or recombinant polypeptide having the amino acid sequence of SEQ ID NO: 5, SEQ ID NO: 27, SEQ ID NO: 28 or SEQ ID NO: 29, being a variant of the amino acid sequence of SEQ ID NO: 5, SEQ ID NO: 27, SEQ ID NO: 28 or SEQ ID NO: 29 having fumonisin amine oxidase activity or a fragment of the amino acid sequence of SEQ ID NO: 5, SEQ ID NO: 27, SEQ ID NO: 28 or SEQ ID NO: 29 having fumonisin amine oxidase activity.

27. The isolated, synthetic or recombinant polypeptide of claim 27, wherein the variant or the fragment has at least 70%, 80%, 90% or 95% identity with respect to the amino acid sequence of SEQ ID NO: 5, SEQ ID NO: 27, SEQ ID NO: 28 or SEQ ID NO: 29.

28. A method for detoxifying a fumonisin mycotoxin, the method comprising contacting the microbial recombinant yeast host cell of any one of claims 1 to 20, the microbial composition of any one of claims 21 to 24, or the isolated, synthetic or recombinant polypeptide of claim 26 or 27 with the fumonisin mycotoxin so as to cause the deamination of the fumonisin mycotoxin into an oxidized fumonisin mycotoxin.

29. The method of claim 28, wherein the fumonisin mycotoxin bears at least one tricarballylic ester substituent.

30. The method of claim 28 or 29 for making a feed product.

31. The method of claim 30, wherein the feed product is or comprises silage, hay, straw, grains, grain by-products, legumes, cottonseed meal, vegetables, milk and/or milk by-products.

32. The method of claim 31, wherein the feed is or comprises grain by-products.

33. The method of claim 32, wherein the grain by-products are distillers grains.

34. The method of claim 28 or 29 for making a food product.

35. The method of claim 34, wherein the food product is or comprises a flour.

36. The method of claim 35, wherein the flour is a corn flour.

37. A feed product comprising the isolated, synthetic or recombinant polypeptide of claim 26 or 27.

38. The feed product of claim 38 further comprising the recombinant microbial host cell of any one of claims 1 to 20 or at least one component from the recombinant microbial host cell of any one of claims 1 to 20.

39. The feed product of claim 37 or 38 being or comprising silage, hay, straw, grains, grain by-products, legumes, cottonseed meal, vegetables, milk and/or milk by-products.

40. The feed of claim 39 being or comprising grain by-products.

41. The feed of claim 40, wherein the grain by-products are distillers grains.

42. The feed product of any one of claims 38 to 41 further comprising an additive.

43. The feed product of claim 43, wherein the additive is a yeast cell wall, a binder or a further mycotoxin-degrading enzyme.

44. A food product comprising the isolated, synthetic or recombinant polypeptide of claim 26 or 27.

45. The food product of claim 44 further comprising the recombinant microbial host cell of any one of claims 1 to 20 or at least one component from the recombinant microbial host cell of any one of claims 1 to 20.

46. The food product of claim 45 being or comprising a flour.

47. The food product of claim 46, wherein the flour is a corn flour.
